(19) United States
(12) Patent Application Publication
(10) Pub. No.: US 2023/0416802 A1
(43) Pub. Date: Dec. 28, 2023

(54) SPECIFIC SUBSTRATES AND PRODUCTS OF HSD17B13 AS MARKERS OF LIVER DISEASE AND BIOMARKERS FOR LIVER DISEASE TREATMENT

(71) Applicant: INIPHARM, INC., Bellevue, WA (US)

(72) Inventors: Heather Kay Webb HSU, Seattle, WA (US); Vincent FLORIO, Seattle, WA (US); Michael CARLETON, Churchville, PA (US); Joshua ODINGO, Bothell, WA (US)

(21) Appl. No.: 18/251,817

(22) PCT Filed: Nov. 3, 2021
(86) PCT No.: PCT/US2021/057895

§ 371 Date: May 4, 2023

Related U.S. Application Data
(60) Provisional application No. 63/110,789, filed on Nov. 6, 2020.

(51) Int. Cl. C12Q 1/32; G01N 33/50

(52) U.S. Cl. CPC C12Q 1/32; C12Y 101/01051; G01N 33/5038

(57) ABSTRACT

Provided are HSD17B13 substrates, reaction products, kits, and methods that include the HSD17B13 substrates or reaction products of the HSD17B13 substrates. Some embodiments include methods of treatment. Some such embodiments include determining an amount of an HSD17B13 substrate or of a reaction product of the HSD17B13 substrate in a sample from the subject to whom the HSD17B13 substrate has been administered.

### CROSS-REFERENCE

This application claims the benefit of U.S. Provisional Application Ser. No. 63/110,789 filed Nov. 6, 2020 which is hereby incorporated by reference in its entirety.

### BACKGROUND

Liver diseases pose a serious problem to many individuals. There is a need for improved treatments and evaluative indices of liver diseases.

### SUMMARY

Provided are HSD17B13 substrates, reaction products, kits, and methods that include the HSD17B13 substrates or reaction products of the HSD17B13 substrates.

Disclosed herein is a method of treatment, comprising: administering to a subject a first amount of a 17β-Hydroxysteroid dehydrogenase type 13 (HSD17B13) substrate or substrate precursor; and determining an amount of a reaction product of the HSD17B13 substrate in a sample obtained from the subject, or determining a second amount of the HSD17B13 substrate or substrate precursor in a sample obtained from the subject, after administering the first amount of the HSD17B13 substrate or substrate precursor to the subject. In some embodiments, the HSD17B13 substrate comprises LTB3, resolvin D1, resolvin E1, protectin DX, maresin 1, 12-HETE, 15-HETE, 13-HODE, 15-HEDE, methyl malonyl coenzyme A, or lauryl coenzyme A, or a salt thereof. In some embodiments, the reaction product comprises an oxidized form of the HSD17B13 substrate. In some embodiments, the HSD17B13 substrate comprises a primary alcohol, and the reaction product comprises an aldehyde. In some embodiments, the HSD17B13 substrate comprises a secondary alcohol, and the reaction product comprises a ketone. In some embodiments, the sample is obtained from the subject about 1 min, about 5 min, about 10 min, about 15 min, about 20 min, about 25 min, about 30 min, about 35 min, about 40 min, about 45 min, about 50 min, about 55 min, about 1 hr, about 2 hr, about 4 hr, about 6 hr, about 8 hr, about 10 hr, about 12 hr, about 14 hr, about 16 hr, about 18 hr, about 20 hr, about 22 hr, about 24 hr, or longer, after administering the first amount of the HSD17B13 substrate or substrate precursor to the subject. In some embodiments, the method further comprises administering or co-administering a first amount of an HSD17B13 inhibitor to the subject with the administration of the first amount of the HSD17B13 substrate or substrate precursor. In some embodiments, the method further comprises determining an HSD17B13 enzyme activity based on the amount of the reaction product, second amount of the HSD17B13 substrate, or second amount of the HSD17B13 substrate precursor in the sample. In some embodiments, the administration of the first amount of the HSD17B13 substrate or substrate precursor is intravenous or oral. In some embodiments, the first amount of the HSD17B13 substrate or substrate precursor is 10-1000 μg of the HSD17B13 substrate or substrate precursor. In some embodiments, the first amount of the HSD17B13 substrate or substrate precursor is about 100 μg of the HSD17B13 substrate or substrate precursor. In some embodiments, the HSD17B13 substrate or substrate precursor and the HSD17B13 reaction product are labeled. In some embodiments, the HSD17B13 substrate or substrate precursor and the HSD17B13 reaction product are radiolabeled. In some embodiments, the radiolabeled HSD17B13 substrate or substrate precursor and reaction product comprise 13C or 2H. In some embodiments, determining the amount of the reaction product comprises measuring the radiolabeled reaction product in the sample. In some embodiments, determining the second amount of the HSD17B13 substrate or substrate precursor comprises measuring the radiolabeled HSD17B13 substrate or substrate precursor in the sample. In some embodiments, the method comprises determining the amount of the reaction product of the HSD17B13 substrate and the second amount of the HSD17B13 substrate or substrate precursor in the sample. In some embodiments, the method further comprises determining a second amount of the reaction product of the HSD17B13 substrate or substrate precursor in a second sample, or determining a third amount of the HSD17B13 substrate or substrate precursor in the second sample, wherein the second sample is obtained from the subject at a second time after administering the first amount of the HSD17B13 substrate to the subject. In some embodiments, the second time comprises about 2 min, about 5 min, about 10 min, about 15 min, about 20 min, about 25 min, about 30 min, about 35 min, about 40 min, about 45 min, about 50 min, about 55 min, about 1 hr, about 2 hr, about 4 hr, about 6 hr, about 8 hr, about 10 hr, about 12 hr, about 14 hr, about 16 hr, about 18 hr, about 20 hr, about 22 hr, about 24 hr, or longer. In some embodiments, the method further comprises comparing the second amount of the reaction product to the first amount of the reaction product, comparing the third amount of the HSD17B13 substrate to the second amount of the HSD17B13 substrate, or comparing the third amount of the HSD17B13 substrate precursor to the second amount of the HSD17B13 substrate precursor. In some embodiments, the method further comprises generating an area under the curve of the first and second amounts of the reaction product, generating an area under the curve of the second and third amounts of the HSD17B13 substrate, or generating an area under the curve of the second and third amounts of the HSD17B13 substrate precursor. In some embodiments, the method further comprises comparing a first area under the curve of the first and second amounts of the reaction product, or the second and third amounts of the HSD17B13 substrate or substrate precursor, to a second area under the curve of amounts of the reaction product, HSD17B13 substrate, or substrate precursor, wherein the first area under the curve is generated when the HSD17B13 substrate or substrate precursor is coadministered to the subject with an HSD17B13 inhibitor, and wherein the second area under the curve is generated when the HSD17B13 substrate or substrate precursor is not coadministered to the subject with the HSD17B13 inhibitor. In some embodiments, the method further comprises comparing a first area under the curve of the first and second amounts of the reaction product, or the second and third amounts of the HSD17B13 substrate or substrate precursor, to a second area under the curve of amounts of the reaction product or HSD17B13 substrate or substrate precursor, wherein the first area under the curve is generated when the HSD17B13 substrate or substrate precursor is coadministered to the subject with a first amount of an HSD17B13 inhibitor, and wherein the second area under the curve is generated when the HSD17B13 substrate or substrate precursor is coadministered to the subject with a second amount of the HSD17B13 inhibitor. In some embodiments, the method further comprises comparing the HSD17B13 enzyme activity to a baseline HSD17B13 enzyme activity obtained without co-administering the HSD17B13 inhibitor to the subject with the HSD17B13 substrate or substrate precursor. In some embodiments, the method further comprises administering a second amount of the HSD17B13 inhibitor to the subject based on the HSD17B13 enzyme activity. In some embodiments, the method further comprises comparing the HSD17B13 enzyme activity to the baseline HSD17B13 enzyme activity, and determining based on the comparison whether to increase, decrease, or not change the second amount of the HSD17B13 inhibitor relative to the first amount of the HSD17B13 inhibitor. In some embodiments, the second amount of the HSD17B13 inhibitor is greater than the first amount of the HSD17B13 inhibitor. In some embodiments, the second amount of the HSD17B13 inhibitor is less than the first amount of the HSD17B13 inhibitor. In some embodiments, the second amount of the HSD17B13 inhibitor is about the same as the first amount of the HSD17B13 inhibitor. In some embodiments, the method further comprises administering, with the first amount of the HSD17B13 substrate or substrate precursor, a first amount of an HSD17B13 inhibitor, wherein the first amount of the HSD17B13 inhibitor inhibits reaction product formation by about 25%, about 50%, about 75%, about 85%, about 90%, or about 95%. In some embodiments, the method further comprises determining the amount of the HSD17B13 inhibitor that inhibits reaction product formation by about 25%, about 50%, about 75%, about 85%, about 90%, or about 95%. In some embodiments, the method further comprises, based on the determined amount of HSD17B13 inhibitor that inhibits reaction product formation by about 25%, about 50%, about 75%, about 85%, about 90%, or about 95%, identifying a dose of the HSD17B13 inhibitor to administer to the subject for treatment. In some embodiments, the dose of the HSD17B13 inhibitor is sufficient to treat a liver disease in the patient. In some embodiments, the method further comprises normalizing the HSD17B13 enzyme activity to obtain a specific HSD17B13 enzyme activity of the subject. In some embodiments, the HSD17B13 enzyme activity is normalized to an amount of the sample to obtain the specific HSD17B13 enzyme activity of the subject. In some embodiments, the method further comprises comparing the HSD17B13 enzyme activity of the subject to a control HSD17B13 enzyme activity measurement, a baseline HSD17B13 enzyme activity measurement, or a threshold HSD17B13 enzyme activity. In some embodiments, the control HSD17B13 enzyme activity measurement is obtained from a general subject population. In some embodiments, the HSD17B13 enzyme activity of the subject is greater than the control HSD17B13 enzyme activity measurement, baseline HSD17B13 enzyme activity measurement, or threshold HSD17B13 enzyme activity. In some embodiments, the method further comprises identifying, based on the HSD17B13 enzyme activity of the subject, the subject as likely to benefit from a treatment with an HSD17B13 inhibitor or as not likely to benefit from the treatment. In some embodiments, the method further comprises identifying the subject as likely to benefit from the treatment with the HSD17B13 inhibitor when the HSD17B13 enzyme activity of the subject is greater than the control HSD17B13 enzyme activity measurement, baseline HSD17B13 enzyme activity measurement, or threshold HSD17B13 enzyme activity. In some embodiments, the HSD17B13 enzyme activity of the subject is about the same or lower than the control HSD17B13 enzyme activity measurement, baseline HSD17B13 enzyme activity measurement, or threshold HSD17B13 enzyme activity. In some embodiments, the method further comprises identifying the subject as unlikely to benefit from the treatment with the HSD17B13 inhibitor when the HSD17B13 enzyme activity of the subject is about the same or lower than the control HSD17B13 enzyme activity measurement, baseline HSD17B13 enzyme activity measurement, or threshold HSD17B13 enzyme activity. In some embodiments, the method further comprises identifying, based on the HSD17B13 enzyme activity measurement, a risk that the subject has or will develop a liver disease. In some embodiments, the method further comprises identifying the subject as likely to develop the liver disease when the HSD17B13 enzyme activity of the subject is greater than the control HSD17B13 enzyme activity measurement, baseline HSD17B13 enzyme activity measurement, or threshold HSD17B13 enzyme activity. In some embodiments, the liver disease comprises liver inflammation, liver fibrosis, cholestasis, a gall bladder disease, a biliary tree disease, alcoholic liver disease, or non-alcoholic steatohepatitis. In some embodiments, the HSD17B13 inhibitor comprises a small molecule, a polypeptide, or an oligonucleotide. In some embodiments, the HSD17B13 inhibitor comprises an estradiol mimetic, propyl pyrazole triole, an anti-HSD17B13 antibody or antibody fragment, an oligonucleotide directed against an HSD17B13 encoding oligonucleotide, an antisense oligonucleotide, an siRNA, ARO-HSD or ALN-HSD. In some embodiments, the HSD17B13 inhibitor is:

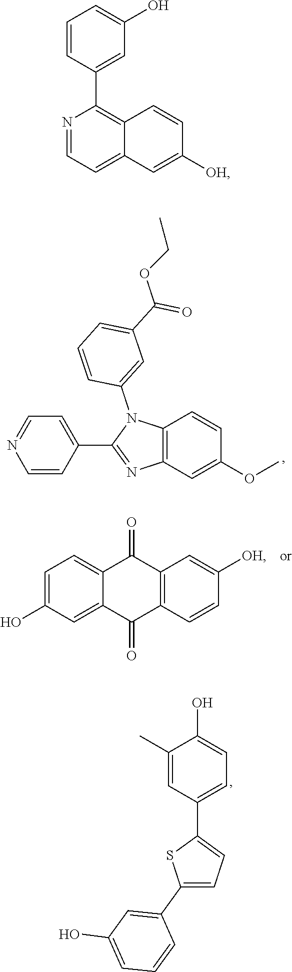

or a pharmaceutically acceptable salt or derivative thereof. In some embodiments, the HSD17B13 inhibitor is:

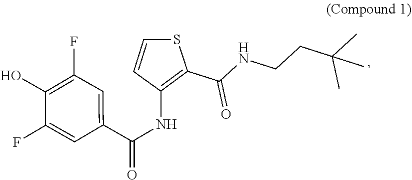 [SMILES: CC(C)(C)CCNC(=O)c1sccc1NC(=O)c1cc(F)c(O)c(F)c1]

or a pharmaceutically acceptable salt or derivative thereof. In some embodiments, measuring the amount of the reaction product or second amount of the HSD17B13 substrate or substrate precursor comprises performing liquid chromatography, high-performance liquid chromatography or mass spectrometry. In some embodiments, the method further comprises administering to the subject the first amount of the HSD17B13 substrate. In some embodiments, the method further comprises determining a second amount of the HSD17B13 substrate in a sample obtained from the subject after administering the first amount of the HSD17B13 substrate to the subject. In some embodiments, the method further comprises administering to the subject the first amount of the HSD17B13 substrate precursor. In some embodiments, the method further comprises determining a second amount of the HSD17B13 substrate precursor in a sample obtained from the subject after administering the first amount of the HSD17B13 substrate precursor to the subject. In some embodiments, the HSD17B13 substrate precursor comprises arachidonic acid or linoleic acid, or a salt thereof. In some embodiments, the sample comprises a blood sample, a plasma sample or a serum sample. In some embodiments, the subject is a mammal. In some embodiments, the subject is a human.

Also disclosed herein is a kit for measuring an HSD17B13 enzyme activity, comprising: a HSD17B13 substrate comprising LTB3, resolvin D1, resolvin E1, protectin DX, maresin 1, 12-HETE, 15-HETE, 13-HODE, 15-HEDE, methyl malonyl coenzyme A or lauryl coenzyme A, or a salt thereof; and a reagent for measuring a reaction product of the HSD17B13 substrate, or a reagent for measuring the HSD17B13 substrate. In some embodiments, the HSD17B13 substrate is labeled. In some embodiments, the HSD17B13 substrate is radiolabeled. In some embodiments, the radiolabeled HSD17B13 substrate comprises 13C or 2H. In some embodiments, the reaction product comprises an oxidized form of the HSD17B13 substrate. In some embodiments, the HSD17B13 substrate comprises a primary alcohol, and the reaction product comprises an aldehyde. In some embodiments, the HSD17B13 substrate comprises a secondary alcohol, and the reaction product comprises a ketone. In some embodiments, the kit further comprises a solution containing a known concentration of HSD17B13. In some embodiments, the kit further comprises instructions for using the reagent in an HSD17B13 activity assay.

Also disclosed herein is an in-vivo modified protein comprising an HSD17B13 enzyme bound to a ligand, wherein the ligand comprises a labeled HSD17B13 substrate comprising LTB3, resolvin D1, resolvin E1, protectin DX, maresin 1, 12-HETE, 15-HETE, 13-HODE, 15-HEDE, methyl malonyl coenzyme A or lauryl coenzyme A, or a salt thereof, or wherein the ligand comprises a labeled reaction product of the HSD17B13 substrate. In some embodiments, the contact comprises a non-covalent bond between the HSD17B13 enzyme and the ligand. In some embodiments, the non-covalent bond comprises a hydrogen bond, an ionic bond, a van der Waals interaction, or a hydrophobic bond. In some embodiments, the HSD17B13 substrate is radiolabeled. In some embodiments, the radiolabeled HSD17B13 substrate comprises 13C or 2H. In some embodiments, the reaction product comprises an oxidized form of the HSD17B13 substrate. In some embodiments, the HSD17B13 substrate comprises a primary alcohol, and the reaction product comprises an aldehyde. In some embodiments, the HSD17B13 substrate comprises a secondary alcohol, and the reaction product comprises a ketone.

Also disclosed herein is a ligand-HSD17B13 complex formed by binding an HSD17B13 enzyme to a ligand, the ligand comprising a labeled HSD17B13 substrate comprising LTB3, resolvin D1, resolvin E1, protectin DX, maresin 1, 12-HETE, 15-HETE, 13-HODE, 15-HEDE, methyl malonyl coenzyme A or lauryl coenzyme A, or a salt thereof, or the ligand comprising a labeled reaction product of the HSD17B13 substrate. In some embodiments, the contact comprises a non-covalent bond between the HSD17B13 enzyme and the ligand. In some embodiments, the non-covalent bond comprises a hydrogen bond, an ionic bond, a van der Waals interaction, or a hydrophobic bond. In some embodiments, the HSD17B13 substrate is radiolabeled. In some embodiments, the radiolabeled HSD17B13 substrate comprises 13C or 2H. In some embodiments, the reaction product comprises an oxidized form of the HSD17B13 substrate. In some embodiments, the HSD17B13 substrate comprises a primary alcohol, and the reaction product comprises an aldehyde. In some embodiments, the HSD17B13 substrate comprises a secondary alcohol, and the reaction product comprises a ketone.

Also disclosed herein is a compound comprising a labeled HSD17B13 substrate comprising LTB3, resolvin D1, resolvin E1, protectin DX, maresin 1, 12-HETE, 15-HETE, 13-HODE, 15-HEDE, methyl malonyl coenzyme A or lauryl coenzyme A, or a salt thereof. In some embodiments, the HSD17B13 substrate is radiolabeled. In some embodiments, the radiolabeled HSD17B13 substrate comprises 13C or 2H.

Also disclosed herein is a compound comprising a reaction product of an HSD17B13 substrate comprising LTB3, resolvin D1, resolvin E1, protectin DX, maresin 1, 12-HETE, 15-HETE, 13-HODE, 15-HEDE, methyl malonyl coenzyme A or lauryl coenzyme A, or a salt thereof. In some embodiments, the reaction product is labeled. In some embodiments, the reaction product is radiolabeled. In some embodiments, the radiolabeled reaction product comprises 13C or 2H. In some embodiments, the reaction product comprises an oxidized form of LTB3, resolvin D1, resolvin E1, protectin DX, maresin 1, 12-HETE, 15-HETE, 13-HODE, 15-HEDE, methyl malonyl coenzyme A or lauryl coenzyme A, or the salt thereof. In some embodiments, the oxidized form of LTB3, resolvin D1, resolvin E1, protectin DX, maresin 1, 12-HETE, 15-HETE, 13-HODE, 15-HEDE, methyl malonyl coenzyme A or lauryl coenzyme A, or the salt thereof comprises an aldehyde or ketone in place of a primary or secondary alcohol of LTB3, resolvin D1, resolvin E1, protectin DX, maresin 1, 12-HETE, 15-HETE, 13-HODE, 15-HEDE, methyl malonyl coenzyme A or lauryl coenzyme A, or the salt thereof.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 includes details of a pharmacophore model of HSD17B13 substrates indicating some similar characteristics.

FIG. 2 is a plot illustrates HSD17B13 readouts in subjects with active HSD17B13, or low or inactive HSD17B13.

FIG. 3A includes plots of labeled substrates and products following intravenous dosing of a subject with an HSD17B13 substrate.

FIG. 3B shows the area under the curve for the product formed to assess percentage inhibition.

FIG. 4 shows the enzyme concentration-dependent oxidation of marker substrate, 13-HODE, by purified HSD17B13 enzyme as measured by NADH production in the presence of 13-HODE as the substrate.

FIG. 5 shows the substrate-concentration-dependent oxidation of marker substrates by purified HSD17B13 enzyme as measured by NADH production in the presence of 13-HODE as the substrate.

FIG. 6 shows the substrate concentration-dependent oxidation of marker substrates, 12-HETE and 15-HEDE, by purified HSD17B13 enzyme as measured by NADH production in the presence of 12-HETE or 15-HEDE as substrates.

FIG. 7 shows substrate-concentration-dependent oxidation of marker substrates by purified HSD17B13 enzyme as measured by NADH production in the presence of LTB3 as the substrate

FIG. 8 shows the maximal activity for resolvin D1, resolvin E1, maresin 1 as compared to LTB3.

FIG. 9 shows the inhibition of NADH formation by purified HSD17B13 in the presence of 15-HEDE and 13-HODE by Compound 1.

FIG. 10 shows the oxidation of several marker substrates in transfected cells.

FIG. 11 shows the inhibition of marker substrate, 12-HETE by Compound 1.

FIG. 12 shows the inhibition of marker substrate, 13-HODE by Compound 1.

### DETAILED DESCRIPTION

Inactive polymorphisms of 17β-Hydroxysteroid dehydrogenase type 13 (HSD17B13) may be protective against chronic liver diseases such as alcoholic liver disease (ALD) and non-alcoholic steatohepatitis (NASH). Thus, HSD17B13 activity is linked to some liver diseases. For this reason, it is useful to measure the extent of HSD17B13 activity in a subject to anticipate their risk of a liver disease such as ALD or NASH. Similarly, to develop inhibitors of HSD17B13, substrates that form specific HSD17B13-dependent products can be used to support dose selection in clinical use. Substrates for HSD17B13 that form unique metabolic products were identified. These substrates can be used to measure the extent that HSD17B13 is inhibited in vivo.

HSD17B13 is an oxido-reductase that oxidizes primary and secondary alcohols on lipophilic molecules. HSD17B13 has been shown to oxidize a few different endogenous molecules including estradiol to form estrone and retinol to form retinaldehyde. Substrates such as these are metabolized to these products by a variety of enzymes, and thus substrate levels and product formation are not useful in measuring HSD17B13 activity in an intact animal for substrates such as estrogen and retinol. The present disclosure solves this problem by identifying several substrates for HSD17B13 whose reaction products with HSD17B13 are not common or are unusual, and are thus useful for obtaining in vivo HSD17B13 enzyme activity levels.

There are numerous polymorphisms for HSD17B13, several of which are catalytically inactive. Inactive HSD17B13 is protective against liver disease, and thus it would be useful to characterize a patient's HSD17B13 activity. Administering HSD17B13 substrates that have specific measurable products will allow for identifying individuals that have active HSD17B13 and thus, higher risk for liver disease.

Individuals with liver disease are likely to benefit from inhibition of HSD17B13. Individuals with liver disease who have active forms of HSD17B13 are especially likely to benefit from inhibition of HSD17B13. To assure selection of clinical doses that are inhibiting the subject's HSD17B13, labeled marker substrates may be provided to the subject with and without dosing of an HSD17B13 inhibitor, and specific product formation may be measured to define inhibitory doses. This can aid in dose selection of an HSD17B13 inhibitor.

Disclosed herein, in some embodiments, are methods such as methods of treating a subject. Some embodiments include administering to a subject a first amount of a 170-Hydroxysteroid dehydrogenase type 13 (HSD17B13) substrate; and determining an amount of a reaction product of the HSD17B13 substrate in a sample obtained from the subject, or determining a second amount of the HSD17B13 substrate in a sample obtained from the subject after administering the first amount of the HSD17B13 substrate to the subject. Some embodiments include administering to a subject a first amount of an HSD17B13 substrate precursor; and determining an amount of the HSD17B13 substrate formed and a reaction product of the HSD17B13 substrate in a sample obtained from the subject, or determining a second amount of the HSD17B13 substrate in a sample obtained from the subject after administering the first amount of the HSD17B13 substrate to the subject.

Disclosed herein, in some embodiments, are kits for measuring an HSD17B13 enzyme activity, comprising: an HSD17B13 substrate comprising LTB3, resolvin D1, resolvin E1, protectin DX, maresin 1, 12-HETE, 15-HETE, 13-HODE, 15-HEDE, methyl malonyl coenzyme A or lauryl coenzyme A, or a salt thereof; and a reagent for measuring a reaction product of the HSD17B13 substrate, or a reagent for measuring the HSD17B13 substrate.

Disclosed herein, in some embodiments, are kits for measuring an HSD17B13 enzyme activity, comprising: the precursor to an HSD17B13 substrate comprising labeled linoleic acid, labeled arachidonic acid or a kit comprising an HSD17B13 substrate comprising LTB3, resolvin D1, resolvin E1, protectin DX, maresin 1, 12-HETE, 15-HETE, 13-HODE, 15-HEDE, methyl malonyl coenzyme A or lauryl coenzyme A, or a salt thereof; and a reagent for measuring a reaction product of the HSD17B13 substrate, or a reagent for measuring the HSD17B 13 substrate.

Disclosed herein, in some embodiments, are kits for measuring an HSD17B13 enzyme activity, comprising: an HSD17B13 substrate comprising LTB3, resolvin D1, resolvin E1, protectin DX, maresin 1, 12-HETE, 13-HODE, 15-HEDE, methyl malonyl coenzyme A or lauryl coenzyme A, or a salt thereof; and a reagent for measuring a reaction product of the HSD17B13 substrate, or a reagent for measuring the HSD17B13 substrate. Also disclosed are in-vivo modified proteins, ligand-HSD17B13 complexes, compounds comprising a labeled HSD17B13 substrate, and compounds comprising a reaction product of an HSD17B13 substrate.

### HSD17B13 Substrates and Reaction Products

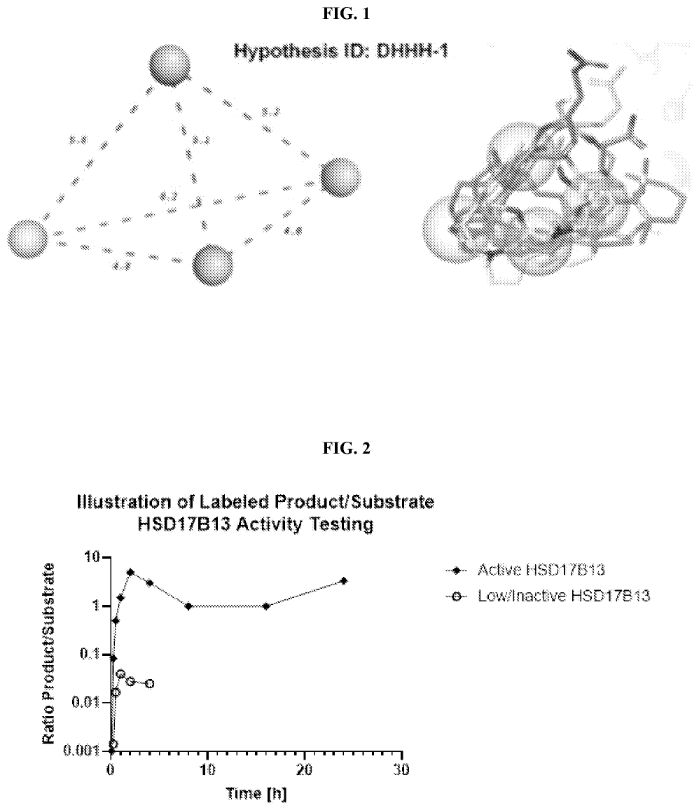

Several hydroxylated lipids were identified as HSD17B13 substrates (Tables 1a and 1b). In Table 1b, Leukotriene B3 (LTB3) is a marker substrate in the hydroxylated lipid class that is used as a comparator. The primary catabolic reaction for these lipids was either incorporation into diglycerides and triglycerides through an acylation reaction, or β-oxidation to form shorter fatty acids. The oxo-products formed by HSD17B13 are not typical molecules found in catabolic pathways, and thus are indicative of HSD17B13 activity. Some of the identified HSD17B13 substrates have precursors that may be useful in the methods and kits described herein. Some such precursors may include arachidonic acid or linoleic acid. For example, arachidonic acid is a precursor to LTB3, 12-HETE and 15-HETE, and linoleic acid is a precursor to 13-HODE. The identified HSD17B13 substrates had features in common that can be defined by a pharmacophore model (FIG. 1; Table 2).

TABLE 1a
HSD17B13 substrates

| HSD17B13 | | |
|---|---|---|
| substrates | Structure | Name |
| LTB3 | 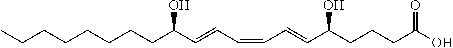 [SMILES: CCCCCCCC[C@@H](O)/C=C/C=C\C=C\[C@@H](O)CCCC(=O)O] | 5S,12R-dihydroxy- 6Z,8E,10E- eicosatrienoic acid |
| Resolvin D1 | 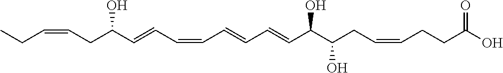 [SMILES: CC/C=C\C[C@H](O)/C=C/C=C\C=C\C=C\[C@@H](O)[C@@H](O)C/C=C\CCC(=O)O] | 7S,8R,17S-trihydroxy- 4Z,9E,11E,13Z,15E,19Z- docosahexaenoic acid |
| Resolvin E1 | 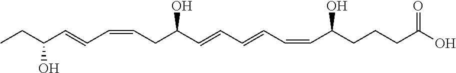 [SMILES: CC[C@@H](O)/C=C/C=C\C[C@@H](O)/C=C/C=C/C=C\[C@@H](O)CCCC(=O)O] | 5S,12R,18R-trihydroxy- 6Z,8E,10E,14Z,16E- eicosapentaenoic acid |
| Protectin DX | 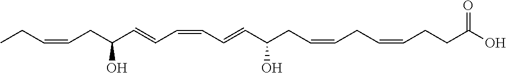 [SMILES: CC/C=C\C[C@H](O)/C=C/C=C\C=C\[C@@H](O)C/C=C\C/C=C\CCC(=O)O] | 10(S),17(S)-dihydroxy- 4Z,7Z,11E,13Z,15E,19Z- docosahexaenoic acid |
| Maresin 1 | 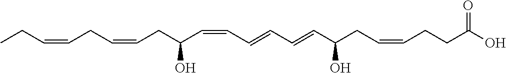 [SMILES: CC/C=C\C/C=C\C[C@H](O)\C=C/C=C/C=C/[C@H](O)C/C=C\CCC(=O)O] | 7R,14S-dihydroxy- 4Z,8E,10E,12Z,16Z,19Z- docosahexaenoic acid |
| (±)12-HETE | 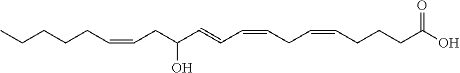 [SMILES: CCCCC/C=C\CC(O)/C=C/C=C\C/C=C\CCCC(=O)O] | (±)12-hydroxy- 5Z,8Z,10E,14Z- eicosatetraenoic acid |
| (±)15-HETE | 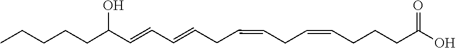 [SMILES: CCCCCC(O)/C=C/C=C/C/C=C\C/C=C\CCCC(=O)O] | (±)15-hydroxy- 5Z,8Z,11Z,13E- eicosatetraenoic acid |
| (±)13-HODE | 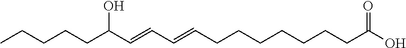 [SMILES: CCCCCC(O)/C=C/C=C/CCCCCCCC(=O)O] | (±)-13-hydroxy-9Z,11E- octadecadienoic acid |
| (±)15-HEDE | 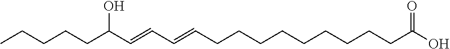 [SMILES: CCCCCC(O)/C=C/C=C/CCCCCCCCCC(=O)O] | (±)-15-hydroxy- 11Z,13E-eicosadienoic acid |
| Methyl malonyl coenzyme A | 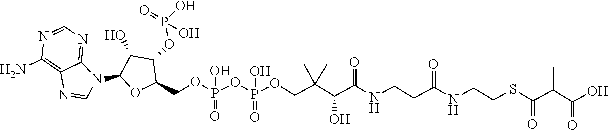 | (9R)-1-[(2R,3S,4R,5R)- 5-(6-Amino-9H-purin- 9-yl)-4-hydroxy-3- (phosphonooxy)oxolan- 2-yl]-3,5,9-trihydroxy- 8,8,20-trimethyl- 3,5,10,14,19-pentaoxo- 2,4,6-trioxa-18-thia- |
| | | 11,15-diaza-3λ5,5λ5- |
| | | diphosphahenicosan-21- |
| | | oic acid |
| Lauroyl coenzyme A | 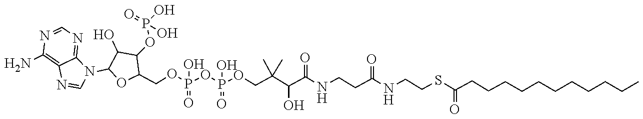 | S-[2-[3-[[(2R)-4- [[[(2R,3S,4R,5R)-5-(6- aminopurin-9-yl)-4- hydroxy-3- phosphonooxyoxolan-2- yl]methoxy- hydroxyphosphoryl]oxy- hydroxyphosphoryl]oxy- |
| | | 2-hydroxy-3,3- |
| | | dimethylbutanoyl]amino] |
| | | propanoylamino]ethyl] |
| | | dodecanethioate |

TABLE 1b
Endogenous hydroxylated lipid HSD17B13 substrates

| Lipid | Relative oxidation at 30 μM (%) |
|---|---|
| Leukotriene B3 (LTB3) | 100 |
| Resolvin D1 | 135 |
| Resolvin E1 | 45 |
| Protectin DX | 105 |
| Maresin 1 | 22 |
| 12(R) HETE | 60 |
| 15 (R) HEDE | 46 |
| Methyl malonyl coenzyme A | 29 (at 10 μM) |
| Lauryl coenzyme A | 35 (AT 3.3 μM) |

TABLE 2

| | | | | | Strain |
|---|---|---|---|---|---|
| | | Site | Vector | Volume | (kcal/ |
| ID | Fitness | Score | Score | Score | mol) |
| Retinol | 0.43 | 0.18 | 0.94 | 0.25 | 0.00 |
| Leukotriene- | 1.10 | 0.73 | 0.78 | 0.37 | 0.00 |
| B3 | | | | | |
| Estradiol | 0.65 | 0.31 | 0.42 | 0.28 | 0.00 |
| 12-HETE | 0.83 | 0.50 | −0.77 | 0.33 | 0.01 |
| 13-HODE | 3.00 | 1.00 | 1.00 | 1.00 | 0.00 |

The pharmacophore illustrated in FIG. 1 provides a 3-dimensional special representation of common features based on an alignment obtained for multiple substrates such as LTB3, estradiol and retinol. This alignment allows for the site of the oxidation to overlap (D) between these differing substrates and allows for the remaining compound bulk to overlap (H). This model can be used to help identify additional substrates through alignment with the 4 common sites of the primary or secondary alcohol in site D and hydrophobic interactions in the remaining 3 H locations spaced per the diagram. The numbers along the lines between the sites are the angstrom distances between the pharmacophore elements.

Disclosed herein, in some embodiments, are HSD17B13 substrates. In some embodiments, the HSD17B13 substrate is a lipid. In some embodiments, the HSD17B13 substrate is hydroxylated. In some embodiments, the HSD17B13 substrate is a hydroxylated lipid.

The HSD17B13 substrate may include LTB3, resolvin D1, resolvin E1, protectin DX, maresin 1, 12-HETE, 15-HETE, 13-HODE, 15-HEDE, methyl malonyl coenzyme A or lauryl coenzyme A, or a salt thereof. In some embodiments, the HSD17B13 substrate includes LTB3, resolvin D1, resolvin E1, protectin DX, maresin 1, 12-HETE, 15-HETE, 13-HODE, 15-HEDE, methyl malonyl coenzyme A, or lauryl coenzyme A. In some embodiments, the HSD17B13 substrate is LTB3, resolvin D1, resolvin E1, protectin DX, maresin 1, 12-HETE, 15-HETE, 13-HODE, 15-HEDE, methyl malonyl coenzyme A, or lauryl coenzyme A. In some embodiments, the HSD17B13 substrate is an endogenous HSD17B13 substrate. In some embodiments, the HSD17B13 substrate includes a salt of LTB3, resolvin D1, resolvin E1, protectin DX, maresin 1, 12-HETE, 15-HETE, 13-HODE, 15-HEDE, methyl malonyl coenzyme A, or lauryl coenzyme A. In some embodiments, the HSD17B13 substrate includes a variant of LTB3, resolvin D1, resolvin E1, protectin DX, maresin 1, 12-HETE, 15-HETE, 13-HODE, 15-HEDE, methyl malonyl coenzyme A, or lauryl coenzyme A. In some embodiments, the variant fits within the pharmacophore model described herein. In some embodiments, the HSD17B13 substrate includes a variant of LTB3, resolvin D1, resolvin E1, protectin DX, maresin 1, 12-HETE, 15-HETE, 13-HODE, 15-HEDE, methyl malonyl coenzyme A, or lauryl coenzyme A, that fits within the pharmacophore model.

The HSD17B13 substrate may include LTB3, resolvin D1, resolvin E1, protectin DX, maresin 1, 12-HETE, 13-HODE, 15-HEDE, methyl malonyl coenzyme A or lauryl coenzyme A, or a salt thereof. In some embodiments, the HSD17B13 substrate includes LTB3, resolvin D1, resolvin E1, protectin DX, maresin 1, 12-HETE, 13-HODE, 15-HEDE, methyl malonyl coenzyme A, or lauryl coenzyme A. In some embodiments, the HSD17B13 substrate is LTB3, resolvin D1, resolvin E1, protectin DX, maresin 1, 12-HETE, 13-HODE, 15-HEDE, methyl malonyl coenzyme A, or lauryl coenzyme A. In some embodiments, the HSD17B13 substrate is an endogenous HSD17B13 substrate. In some embodiments, the HSD17B13 substrate includes a salt of LTB3, resolvin D1, resolvin E1, protectin DX, maresin 1, 12-HETE, 13-HODE, 15-HEDE, methyl malonyl coenzyme A, or lauryl coenzyme A. In some embodiments, the HSD17B13 substrate includes a variant of LTB3, resolvin D1, resolvin E1, protectin DX, maresin 1, 12-HETE, 13-HODE, 15-HEDE, methyl malonyl coenzyme A, or lauryl coenzyme A. In some embodiments, the variant fits within the pharmacophore model described herein. In some embodiments, the HSD17B13 substrate includes a variant of LTB3, resolvin D1, resolvin E1, protectin DX, maresin 1, 12-HETE, 13-HODE, 15-HEDE, methyl malonyl coenzyme A, or lauryl coenzyme A, that fits within the pharmacophore model.

In some embodiments, the HSD17B13 substrate includes LTB3. In some embodiments, the HSD17B13 substrate includes LTB3, or a salt thereof.

In some embodiments, the HSD17B13 substrate includes resolvin D1. In some embodiments, the HSD17B13 substrate includes resolvin D1, or a salt thereof.

In some embodiments, the HSD17B13 substrate includes resolvin E1. In some embodiments, the HSD17B13 substrate includes resolvin E1, or a salt thereof.

In some embodiments, the HSD17B13 substrate includes protectin DX. In some embodiments, the HSD17B13 substrate includes protectin DX, or a salt thereof.

In some embodiments, the HSD17B13 substrate includes maresin 1. In some embodiments, the HSD17B13 substrate includes maresin 1, or a salt thereof.

In some embodiments, the HSD17B13 substrate includes 15-HETE. In some embodiments, the HSD17B13 substrate includes 15-HETE, or a salt thereof. The 15-HETE may include 15(R)-HETE. The 15-HETE may include 15(S)-HETE. The 15-HETE may include an entaniomeric mixture of 15(R)-HETE and 15(S)-HETE.

In some embodiments, the HSD17B13 substrate includes 12-HETE. In some embodiments, the HSD17B13 substrate includes 12-HETE, or a salt thereof. The 12-HETE may include 12(R)-HETE. The 12-HETE may include 12(S)-HETE. The 12-HETE may include an entaniomeric mixture of 12(R)-HETE and 12(S)-HETE.

In some embodiments, the HSD17B13 substrate includes 13-HODE. In some embodiments, the HSD17B13 substrate includes 13-HODE, or a salt thereof. The 13-HODE may include 13(R)-HODE. The 13-HODE may include 13(S)-HODE. The 13-HODE may include an entaniomeric mixture of 13(R)-HODE and 13(S)-HODE.

In some embodiments, the HSD17B13 substrate includes 15-HEDE In some embodiments, the HSD17B13 substrate includes 15-HEDE, or a salt thereof. The 15-HEDE may include 15(R)-HEDE. The 15-HEDE may include 15(S)-HEDE. The 15-HEDE may include an entaniomeric mixture of 15(R)-HEDE and 15(S)-HEDE.

In some embodiments, the HSD17B13 substrate includes methyl malonyl coenzyme A. In some embodiments, the HSD17B13 substrate includes methyl malonyl coenzyme A, or a salt thereof.

In some embodiments, the HSD17B13 substrate includes lauryl coenzyme A. In some embodiments, the HSD17B13 substrate includes lauryl coenzyme A, or a salt thereof.

The HSD17B13 substrates can be formed in cells, animals and humans by administering a substrate precursor such as arachidonic acid or linoleic acid. Some embodiments relate to an HSD17B13 substrate precursor. Examples of substrate precursors include arachidonic acid and linoleic acid.

In some embodiments, the HSD17B13 substrate precursor includes arachidonic acid. In some embodiments, the HSD17B13 substrate precursor includes arachidonic acid, or a salt thereof. In some embodiments, the HSD17B13 precursor comprises arachidonic acid or a labeled arachidonic acid or salt thereof.

In some embodiments, the HSD17B13 substrate precursor includes linoleic acid. In some embodiments, the HSD17B13 substrate precursor includes linoleic acid, or a salt thereof. In some embodiments, the HSD17B13 precursor comprises linoleic acid or a labeled linoleic acid or salt thereof.

In some embodiments, the HSD17B13 substrate or substrate precursor is labeled. In some embodiments, the HSD17B13 substrate is labeled. The label may include a radiolabel. For example, the LTB3, resolvin D1, resolvin E1, protectin DX, maresin 1, 12-HETE, 15-HETE, 13-HODE, 15-HEDE, methyl malonyl coenzyme A, or lauryl coenzyme A may be radiolabeled. An example of a radiolabel includes 2H. An example of a radiolabel includes 2H. One or both of these radiolabels may be used. Other labels or radiolabels may be used. In some embodiments, the LTB3, resolvin D1, resolvin E1, protectin DX, maresin 1, 12-HETE, 15-HETE, 13-HODE, 15-HEDE, methyl malonyl coenzyme A, or lauryl coenzyme A, comprises 13C or 2H.

In some embodiments, the HSD17B13 substrate is labeled. The label may include a radiolabel. For example, the LTB3, resolvin D1, resolvin E1, protectin DX, maresin 1, 12-HETE, 13-HODE, 15-HEDE, methyl malonyl coenzyme A, or lauryl coenzyme A may be radiolabeled. An example of a radiolabel includes 2H. An example of a radiolabel includes 2H. One or both of these radiolabels may be used. Other labels or radiolabels may be used. In some embodiments, the LTB3, resolvin D1, resolvin E1, protectin DX, maresin 1, 12-HETE, 13-HODE, 15-HEDE, methyl malonyl coenzyme A, or lauryl coenzyme A, comprises 13C or 2H.

In some embodiments, the HSD17B13 substrate precursor is labeled. The label may include a radiolabel. For example, the arachidonic acid or linoleic acid may be radiolabeled. The radiolabel of the arachidonic acid or linoleic acid may include 13C or 2H. The radiolabel of the arachidonic acid or linoleic acid may include 13C. The radiolabel of the arachidonic acid or linoleic acid may include 2H.

Disclosed herein, in some embodiments, are reaction products of HSD17B13 substrates. The HSD17B13 may react with the HSD17B13 substrate to form an HSD17B13 reaction product. The HSD17B13 reaction product may include a lipid. The HSD17B13 reaction product may include a hydroxylated lipid reacted with HSD17B13.

HSD17B13 substrate reaction products include HSD17B13 substrates that have been reacted with HSD17B13. HSD17B13 may oxidize its substrates at a primary or secondary alcohol to form an aldehyde or ketone, respectively. Examples of reaction products of HSD17B13 substrates include reaction products of LTB3, resolvin D1, resolvin E1, protectin DX, maresin 1, 12-HETE, 15-HETE, 13-HODE, 15-HEDE, methyl malonyl coenzyme A, or lauryl coenzyme A. Examples of reaction products of HSD17B13 substrates include reaction products of LTB3, resolvin D1, resolvin E1, protectin DX, maresin 1, 12-HETE, 15-HETE, 13-HODE, 15-HEDE, methyl malonyl coenzyme A, or lauryl coenzyme A, or a salt thereof. The reaction product may be labeled. In some embodiments, the reaction product is radiolabeled. In some embodiments, the radiolabeled reaction product comprises 13C or 2H. In some embodiments, the radiolabeled reaction product comprises 13C. In some embodiments, the radiolabeled reaction product comprises 2H.

HSD17B13 substrate reaction products include HSD17B13 substrates that have been reacted with HSD17B13. HSD17B13 may oxidize its substrates at a primary or secondary alcohol to form an aldehyde or ketone, respectively. Examples of reaction products of HSD17B13 substrates include reaction products of LTB3, resolvin D1, resolvin E1, protectin DX, maresin 1, 12-HETE, 13-HODE, 15-HEDE, methyl malonyl coenzyme A, or lauryl coenzyme A. Examples of reaction products of HSD17B13 substrates include reaction products of LTB3, resolvin D1, resolvin E1, protectin DX, maresin 1, 12-HETE, 13-HODE, 15-HEDE, methyl malonyl coenzyme A, or lauryl coenzyme A, or a salt thereof. The reaction product may be labeled. In some embodiments, the reaction product is radiolabeled. In some embodiments, the radiolabeled reaction product comprises 13C or 2H. In some embodiments, the radiolabeled reaction product comprises 13C. In some embodiments, the radiolabeled reaction product comprises 2H.

Some embodiments include a reaction product comprising an oxidized form of LTB3. Some embodiments include a reaction product comprising an oxidized form of resolvin D1. Some embodiments include a reaction product comprising an oxidized form of resolvin E1. Some embodiments include a reaction product comprising an oxidized form of protectin DX. Some embodiments include a reaction product comprising an oxidized form of 15-HETE.

Some embodiments include a reaction product comprising an oxidized form of maresin 1. Some embodiments include a reaction product comprising an oxidized form of 12-HETE. Some embodiments include a reaction product comprising an oxidized form of 13-HODE. Some embodiments include a reaction product comprising an oxidized form of 15-HEDE. Some embodiments include a reaction product comprising an oxidized form of methyl malonyl coenzyme A. Some embodiments include a reaction product comprising an oxidized form of lauryl coenzyme A.

### In Vivo HSD17B13 Activity and Related Methods

Disclosed herein, in some embodiments, are methods such as methods of treating a subject. The methods may include a 17β-Hydroxysteroid dehydrogenase type 13 (HSD17B13) activity described herein. The methods may be used to identify a dose of an HSD17B13 inhibitor to administer to a subject, determine whether a subject is likely to benefit from treatment with an HSD17B13 inhibitor, or determine a subject's risk of developing a liver disease.

Some embodiments include administering an HSD17B13 substrate to a subject. Some embodiments include administering to a subject a first amount of an HSD17B13 substrate. Some embodiments include determining an amount of a reaction product of the HSD17B13 substrate. Some embodiments include determining an amount of a reaction product of the HSD17B13 substrate in a sample obtained from the subject. In some embodiments, the sample is obtained from the subject after administering the first amount of the HSD17B13 substrate to the subject. Some embodiments include determining a second amount of the HSD17B13 substrate. Some embodiments include determining a second amount of the HSD17B13 substrate in a sample obtained from the subject. In some embodiments, the sample is obtained from the subject after administering the first amount of the HSD17B13 substrate to the subject. Some embodiments include determining an amount of a reaction product of the HSD17B13 substrate and the second amount of the HSD17B13 substrate in the same sample. Some embodiments include determining an amount of a reaction product of the HSD17B13 substrate and the second amount of the HSD17B13 substrate in separate samples. Disclosed herein, in some embodiments, are methods of treatment, comprising: administering to a subject a first amount of an HSD17B13 substrate; and determining an amount of a reaction product of the HSD17B13 substrate in a sample obtained from the subject, or determining a second amount of the HSD17B13 substrate in a sample obtained from the subject after administering the first amount of the HSD17B13 substrate to the subject.

Some embodiments include administering an HSD17B13 substrate precursor to a subject. Some embodiments include administering to a subject a first amount of an HSD17B13 substrate precursor. Some embodiments include determining an amount of the HSD17B13 substrate before administering the HSD17B13 substrate precursor to a subject. Some embodiments include determining an amount of the HSD17B13 substrate after administering the HSD17B13 substrate precursor to a subject. Some embodiments include determining an amount of a reaction product of the HSD17B13 substrate. Any of the determinations (e.g. measurements) may be made in a sample obtained from the subject. A sample for measurement may be obtained from the subject before or after administering the first amount of the HSD17B13 substrate precursor to the subject. Some embodiments include determining a second amount of the HSD17B13 substrate precursor. Some embodiments include determining a second amount of the HSD17B13 substrate precursor in a sample obtained from the subject. In some embodiments, the sample is obtained from the subject after administering the first amount of the HSD17B13 substrate precursor to the subject. Some embodiments include administering to a subject a first amount of an HSD17B13 substrate precursor; determining an amount of the HSD17B13 substrate formed; and determining an amount of a reaction product of the HSD17B13 substrate in a sample obtained from the subject. Some embodiments include determining a second amount of the HSD17B13 substrate precursor in a sample obtained from the subject after administering the first amount of the HSD17B13 substrate precursor to the subject.

In some embodiments, the subject has already been administered the HSD17B13 substrate (or substrate precursor). Some such embodiments include determining an amount of a reaction product of the HSD17B13 substrate in a sample obtained from a subject that has been administered an HSD17B13 substrate. Some such embodiments include determining a second amount of the HSD17B13 substrate in a sample obtained from a subject that has been administered a first amount of an HSD17B13 substrate.

The substrate may include an HSD17B13 substrate described herein. In some embodiments, the HSD17B13 substrate comprises LTB3, resolvin D1, resolvin E1, protectin DX, maresin 1, 12-HETE, 15-HETE, 13-HODE, 15-HEDE, methyl malonyl coenzyme A, or lauryl coenzyme A, or a salt thereof. The reaction product may include an HSD17B13 substrate described herein. The substrate may include an HSD17B13 substrate described herein. In some embodiments, the HSD17B13 substrate comprises LTB3, resolvin D1, resolvin E1, protectin DX, maresin 1, 12-HETE, 13-HODE, 15-HEDE, methyl malonyl coenzyme A, or lauryl coenzyme A, or a salt thereof. The reaction product may include an HSD17B13 substrate described herein. In some embodiments, the reaction product comprises an oxidized form of the HSD17B13 substrate. In some embodiments, the HSD17B13 substrate comprises a primary alcohol, and the reaction product comprises an aldehyde. In some embodiments, the HSD17B13 substrate comprises a secondary alcohol, and the reaction product comprises a ketone.

Some embodiments include obtaining a sample or a first sample from a subject. In some embodiments, the sample is obtained from the subject 1 min, 5 min, 10 min, 15 min, 20 min, 25 min, 30 min, 35 min, 40 min, 45 min, 50 min, 55 min, 1 hr, 2 hr, 4 hr, 6 hr, 8 hr, 10 hr, 12 hr, 14 hr, 16 hr, 18 hr, 20 hr, 22 hr, or 24 hr, or a range of times defined by any two of the aforementioned times, after administering the first amount of the HSD17B13 substrate to the subject. In some embodiments, the sample is obtained from the subject at least 1 min, at least 5 min, at least 10 min, at least 15 min, at least 20 min, at least 25 min, at least 30 min, at least 35 min, at least 40 min, at least 45 min, at least 50 min, at least 55 min, at least 1 hr, at least 2 hr, at least 4 hr, at least 6 hr, at least 8 hr, at least 10 hr, at least 12 hr, at least 14 hr, at least 16 hr, at least 18 hr, at least 20 hr, at least 22 hr, at least or at least 24 hr, after administering the first amount of the HSD17B13 substrate to the subject. In some embodiments, the sample is obtained from the subject no more than 1 min, no more than 5 min, no more than 10 min, no more than 15 min, no more than 20 min, no more than 25 min, no more than 30 min, no more than 35 min, no more than 40 min, no more than 45 min, no more than 50 min, no more than 55 min, no more than 1 hr, no more than 2 hr, no more than 4 hr, no more than 6 hr, no more than 8 hr, no more than 10 hr, no more than 12 hr, no more than 14 hr, no more than 16 hr, no more than 18 hr, no more than 20 hr, no more than 22 hr, no more than or no more than 24 hr, after administering the first amount of the HSD17B13 substrate to the subject. In some embodiments, the sample is obtained from the subject about 1 min, about 5 min, about 10 min, about 15 min, about 20 min, about 25 min, about 30 min, about 35 min, about 40 min, about 45 min, about 50 min, about 55 min, about 1 hr, about 2 hr, about 4 hr, about 6 hr, about 8 hr, about 10 hr, about 12 hr, about 14 hr, about 16 hr, about 18 hr, about 20 hr, about 22 hr, about 24 hr, or longer, after administering the first amount of the HSD17B13 substrate to the subject.

Some embodiments include administration or coadministration of an HSD17B13 inhibitor to the subject with or at a similar time as the HSD17B13 substrate. Some embodiments include administering or co-administering a first amount of an HSD17B13 inhibitor to the subject with the administration of the first amount of the HSD17B13 substrate. The administration or coadministration of an HSD17B13 inhibitor may be used to determine an amount by which the HSD17B13 inhibitor inhibits HSD17B13 activity in the subject. This may be useful for obtaining the correct dosing of the subject of the HSD17B13 inhibitor.

Some embodiments include determining an HSD17B13 enzyme activity. The HSD17B13 enzyme activity may be determined based on the amount of the reaction product measured in the sample obtained after administration of the HSD17B13 substrate to the subject. The HSD17B13 enzyme activity may be determined based on the amount of HSD17B13 substrate measured in the sample obtained after administration of the HSD17B13 substrate to the subject. Some embodiments include determining an HSD17B13 enzyme activity based on the amount of the reaction product or second amount of the HSD17B13 substrate in the sample. Such measurements may be made over time, and an area under the curve may be obtained.

Some embodiments include administering an HSD17B13 substrate (such as a first amount of the HSD17B13 substrate) to a subject. In some embodiments, the administration of the HSD17B13 substrate to the subject is intravenous. For example, the HSD17B13 substrate may be infused into the bloodstream of the subject. The HSD17B13 substrate may be injected into the subject or into the subject's bloodstream. In some embodiments, the administration of the HSD17B13 substrate to the subject is oral. In some embodiments, the administration of the first amount of the HSD17B13 substrate is intravenous or oral.

Some embodiments include administering an amount of an HSD17B13 substrate (such as a first amount of the HSD17B13 substrate) to a subject. Various amounts of the HSD17B13 substrate may be administered. For example, micro amounts may be administered to the subject. In some embodiments, 5 μg, 10 μg, 25 μg, 50 μg, 75 μg, 100 μg, 150 μg, 200 μg, 250 μg, 500 μg, 750 μg, or 1000 μg, or a range defined by any of the aforementioned amounts, of the HSD17B13 substrate is administered to the subject. In some embodiments, about 5 μg, about 10 μg, about 25 μg, about 50 μg, about 75 μg, about 100 μg, about 150 μg, about 200 μg, about 250 μg, about 500 μg, about 750 μg, or about 1000 μg, of the HSD17B13 substrate is administered to the subject. In some embodiments, at least 5μg, at least 10 μg, at least 25 μg, at least 50 μg, at least 75 μg, at least 100 μg, at least 150 μg, at least 200 μg, at least 250 μg, at least 500 μg, at least 750 μg, or at least 1000 μg, of the HSD17B13 substrate is administered to the subject. In some embodiments, no more than 5 μg, no more than 10 μg, no more than 25 μg, no more than 50 μg, no more than 75 μg, no more than 100 μg, no more than 150 μg, no more than 200 μg, no more than 250 μg, no more than 500 μg, no more than 750 μg, or no more than 1000 μg, of the HSD17B13 substrate is administered to the subject. In some embodiments, the first amount of the HSD17B13 substrate is 10-1000 i.tg of the HSD17B13 substrate. In some embodiments, the first amount of the HSD17B13 substrate is about 100 μg of the HSD17B13 substrate.

In some embodiments, the HSD17B13 substrate and the HSD17B13 reaction product are labeled. In some embodiments, the HSD17B13 substrate and the HSD17B13 reaction product are radiolabeled. In some embodiments, the radiolabeled HSD17B13 substrate and reaction product comprise 13C or 2H. In some embodiments, the HSD17B13 substrate is labeled. In some embodiments, the HSD17B13 substrate is radiolabeled. In some embodiments, the radiolabeled HSD17B13 substrate comprises 13C. In some embodiments, the radiolabeled HSD17B13 substrate comprises 2H. In some embodiments, the radiolabeled HSD17B13 substrate comprises 13C and 2H. In some embodiments, determining the second amount of the HSD17B13 substrate comprises measuring the radiolabeled HSD17B13 substrate in the sample. In some embodiments, the HSD17B13 reaction product is labeled. In some embodiments, the HSD17B13 reaction product is radiolabeled. In some embodiments, the radiolabeled HSD17B13 reaction product comprises 13C. In some embodiments, the radiolabeled HSD17B13 reaction product comprises 2H. In some embodiments, the radiolabeled HSD17B13 reaction product comprises 13C and 2H. In some embodiments, determining the amount of the reaction product comprises measuring the radiolabeled reaction product in the sample.

In some embodiments, identifying the HSD17B13 enzyme activity based on the amount of the reaction product or second amount of the HSD17B13 substrate in the sample, comprises detecting increases in oxidized 13C or 2H labeled product over time after the administration of the first amount of the HSD17B13 substrate, or comprises detecting decreases in 13C or 2H labeled substrate over time after the administration of the first amount of the HSD17B13 substrate. In some embodiments, identifying the HSD17B13 enzyme activity based on the amount of the reaction product in the sample, comprises detecting increases in oxidized 13C or 2H labeled product over time after the administration of the first amount of the HSD17B13 substrate. In some embodiments, identifying the HSD17B13 enzyme activity based on the second amount of the HSD17B13 substrate in the sample, comprises detecting decreases in 13C or 2H labeled substrate over time after the administration of the first amount of the HSD17B 13 substrate.

Some embodiments include determining the amount of the reaction product of the HSD17B13 substrate in the sample. Some embodiments include determining the second amount of the HSD17B13 substrate in the sample. Some embodiments include determining the amount of the reaction product of the HSD17B13 substrate and the second amount of the HSD17B13 substrate in the sample.

Some embodiments include determining a second amount of the reaction product of the HSD17B13 substrate in a second sample. Some embodiments include determining a third amount of the HSD17B13 substrate in the second sample. In some embodiments, the second sample is obtained from the subject at a second time after administering the first amount of the HSD17B13 substrate to the subject. Some embodiments include determining a second amount of the reaction product of the HSD17B13 substrate in a second sample, or determining a third amount of the HSD17B13 substrate in the second sample, wherein the second sample is obtained from the subject at a second time after administering the first amount of the HSD17B13 substrate to the subject.

In some embodiments, the second time comprises 2 min, 5 min, 10 min, 15 min, 20 min, 25 min, 30 min, 35 min, 40 min, 45 min, 50 min, 55 min, 1 hr, 2 hr, 4 hr, 6 hr, 8 hr, 10 hr, 12 hr, 14 hr, 16 hr, 18 hr, 20 hr, 22 hr, or 24 hr, or a range of time periods defined by any two of the aforementioned time periods. In some embodiments, the second time comprises at least 2 min, at least 5 min, at least 10 min, at least 15 min, at least 20 min, at least 25 min, at least 30 min, at least 35 min, at least 40 min, at least 45 min, at least 50 min, at least 55 min, at least 1 hr, at least 2 hr, at least 4 hr, at least 6 hr, at least 8 hr, at least 10 hr, at least 12 hr, at least 14 hr, at least 16 hr, at least 18 hr, at least 20 hr, at least 22 hr, or at least 24 hr. In some embodiments, the second time comprises no more than 2 min, no more than 5 min, no more than 10 min, no more than 15 min, no more than 20 min, no more than 25 min, no more than 30 min, no more than 35 min, no more than 40 min, no more than 45 min, no more than 50 min, no more than 55 min, no more than 1 hr, no more than 2 hr, no more than 4 hr, no more than 6 hr, no more than 8 hr, no more than 10 hr, no more than 12 hr, no more than 14 hr, no more than 16 hr, no more than 18 hr, no more than 20 hr, no more than 22 hr, or no more than 24 hr. In some embodiments, the second time comprises about 2 min, about 5 min, about 10 min, about 15 min, about 20 min, about 25 min, about 30 min, about 35 min, about 40 min, about 45 min, about 50 min, about 55 min, about 1 hr, about 2 hr, about 4 hr, about 6 hr, about 8 hr, about 10 hr, about 12 hr, about 14 hr, about 16 hr, about 18 hr, about 20 hr, about 22 hr, or about 24 hr, or longer.

Some embodiments include comparing the second amount of the reaction product to the first amount of the reaction product. Some embodiments include comparing the third amount of the HSD17B13 substrate to the second amount of the HSD17B13 substrate. Some embodiments include comparing the second amount of the reaction product to the first amount of the reaction product, or comparing the third amount of the HSD17B13 substrate to the second amount of the HSD17B13 substrate.

Some embodiments include generating an area under the curve of the first and second amounts of the reaction product. Some embodiments include generating an area under the curve of the second and third amounts of the HSD17B13 substrate. Some embodiments include generating an area under the curve of the first and second amounts of the reaction product, or generating an area under the curve of the second and third amounts of the HSD17B13 substrate.

Some embodiments include comparing a first area under the curve of the first and second amounts of the reaction product. Some embodiments include comparing a first area under the curve of the first and second amounts of the reaction product to a second area under the curve of amounts of the reaction product or HSD17B13 substrate. Some embodiments include comparing the second and third amounts of the HSD17B13 substrate. Some embodiments include comparing the second and third amounts of the HSD17B13 substrate to a second area under the curve of amounts of the reaction product or HSD17B13 substrate. In some embodiments, the first area under the curve is generated when the HSD17B13 substrate is coadministered to the subject with an HSD17B13 inhibitor. In some embodiments, the second area under the curve is generated when the HSD17B13 substrate is not coadministered to the subject with the HSD17B13 inhibitor. Some embodiments include comparing a first area under the curve of the first and second amounts of the reaction product, or the second and third amounts of the HSD17B13 substrate, to a second area under the curve of amounts of the reaction product or HSD17B13 substrate, wherein the first area under the curve is generated when the HSD17B13 substrate is coadministered to the subject with an HSD17B13 inhibitor, and wherein the second area under the curve is generated when the HSD17B13 substrate is not coadministered to the subject with the HSD17B13 inhibitor. In some embodiments, the first area under the curve is generated when the HSD17B13 substrate is coadministered to the subject with a first amount of an HSD17B13 inhibitor. In some embodiments, the second area under the curve is generated when the HSD17B13 substrate is coadministered to the subject with a second amount of the HSD17B13 inhibitor. Some embodiments include comparing a first area under the curve of the first and second amounts of the reaction product, or the second and third amounts of the HSD17B13 substrate, to a second area under the curve of amounts of the reaction product or HSD17B13 substrate, wherein the first area under the curve is generated when the HSD17B13 substrate is coadministered to the subject with a first amount of an HSD17B13 inhibitor, and wherein the second area under the curve is generated when the HSD17B13 substrate is coadministered to the subject with a second amount of the HSD17B13 inhibitor.

Some embodiments include comparing the HSD17B13 enzyme activity to a baseline HSD17B13 enzyme activity obtained without co-administering the HSD17B13 inhibitor to the subject with the HSD17B13 substrate. Some embodiments include administering a second amount of the HSD17B13 inhibitor to the subject based on the HSD17B13 enzyme activity. Some embodiments include comparing the HSD17B13 enzyme activity to the baseline HSD17B13 enzyme activity. Some embodiments include determining based on the comparison whether to increase, decrease, or not change the second amount of the HSD17B13 inhibitor relative to the first amount of the HSD17B13 inhibitor. Some embodiments include comparing the HSD17B13 enzyme activity to the baseline HSD17B13 enzyme activity, and determining based on the comparison whether to increase, decrease, or not change the second amount of the HSD17B13 inhibitor relative to the first amount of the HSD17B13 inhibitor. In some embodiments, the second amount of the HSD17B13 inhibitor is greater than the first amount of the HSD17B13 inhibitor. In some embodiments, the second amount of the HSD17B13 inhibitor is less than the first amount of the HSD17B13 inhibitor. In some embodiments, the second amount of the HSD17B13 inhibitor is about the same as the first amount of the HSD17B13 inhibitor.

Some embodiments include providing the subject with a 13C or 2H labeled HSD17B13 substrate with different doses of the HSD17B13 inhibitor to obtain dose levels that inhibit labeled product formation at 25%, 50%, 75%, 85%, 90%, or 95%. Some embodiments include administering a labeled HSD17B13 substrate to a subject. The subject may also be coadministered one or more doses of an HSD17B13 inhibitor. Some embodiments include determining what amounts of inhibition each of the one or more doses (e.g. multiple doses) has upon HSD17B13 activity within the subject. The HSD17B13 activity may be determined at each dose of the HSD17B13 inhibitor. Thus, the optimal dose may be provided based on what amount each dose inhibits HSD17B13 activity, and what amount of HSD17B13 activity inhibition is desired.

Some embodiments include performing assays to aid in selection of clinical doses that sufficiently inhibit the subject's HSD17B13. For example, some embodiments include administering, with the first amount of the HSD17B13 substrate, a first amount of an HSD17B13 inhibitor, wherein the first amount of the HSD17B13 inhibitor inhibits reaction product formation by about 25%, about 50%, about 75%, about 85%, about 90%, or about 95%. Some embodiments include administering, with the first amount of the HSD17B13 substrate, a first amount of an HSD17B13 inhibitor. In some embodiments, the first amount of the HSD17B13 inhibitor inhibits reaction product formation by an amount. Some embodiments include administering, with the first amount of the HSD17B13 substrate, a first amount of an HSD17B13 inhibitor, wherein the first amount of the HSD17B13 inhibitor inhibits reaction product formation by an amount. In some embodiments, the first amount of the HSD17B13 inhibitor inhibits reaction product formation by 25%, 50%, 75%, 85%, 90%, or 95%, or a range of percentages defined by any two of the aforementioned percentages. In some embodiments, the first amount of the HSD17B13 inhibitor inhibits reaction product formation by about 25%, about 50%, about 75%, about 85%, about 90%, or about 95%. In some embodiments, the first amount of the HSD17B13 inhibitor inhibits reaction product formation by at least 25%, at least 50%, at least 75%, at least 85%, at least 90%, or at least 95%. In some embodiments, the first amount of the HSD17B13 inhibitor inhibits reaction product formation by no greater than 25%, no greater than 50%, no greater than 75%, no greater than 85%, no greater than 90%, or no greater than 95%.

Some embodiments include determining the amount of the HSD17B13 inhibitor that inhibits reaction product formation by a desired amount (such as about 25%, about 50%, about 75%, about 85%, about 90%, or about 95%). In some cases, the desired amount is at least 25%, at least 50%, at least 75%, at least 85%, at least 90%, or at least 95%. In some cases, the desired amount is no greater than 25%, no greater than 50%, no greater than 75%, no greater than 85%, no greater than 90%, or no greater than 95%. Some embodiments include determining the amount of the HSD17B13 inhibitor that inhibits reaction product formation by about 25%, about 50%, about 75%, about 85%, about 90%, or about 95%. Some embodiments include, based on the determined amount of HSD17B13 inhibitor that inhibits reaction product formation by the desired amount, identifying a dose of the HSD17B13 inhibitor to administer to the subject for treatment. Some embodiments include, based on the determined amount of HSD17B13 inhibitor that inhibits reaction product formation by about 25%, about 50%, about 75%, about 85%, about 90%, or about 95%, identifying a dose of the HSD17B13 inhibitor to administer to the subject for treatment. These methods may enable a clinician to recommend or give the correct dose of an HSD17B13 inhibitor to the subject. In some embodiments, the dose of the HSD17B13 inhibitor is sufficient to treat a liver disease in the patient.

Some embodiments include normalizing the HSD17B13 enzyme activity to obtain a specific HSD17B13 enzyme activity of the subject. In some embodiments, the HSD17B13 enzyme activity is normalized to an amount of the sample to obtain the specific HSD17B13 enzyme activity of the subject. Some embodiments include comparing the HSD17B13 enzyme activity of the subject to a control HSD17B13 enzyme activity measurement, a baseline HSD17B13 enzyme activity measurement, or a threshold HSD17B13 enzyme activity. Some embodiments include comparing the HSD17B13 enzyme activity of the subject to a control HSD17B13 enzyme activity measurement. Some embodiments include comparing the HSD17B13 enzyme activity of the subject to a baseline HSD17B13 enzyme activity measurement. Some embodiments include comparing the HSD17B13 enzyme activity of the subject to a threshold HSD17B13 enzyme activity. In some embodiments, the control HSD17B13 enzyme activity measurement is obtained from a general subject population. In some embodiments, the HSD17B13 enzyme activity of the subject is greater than the control HSD17B13 enzyme activity measurement, baseline HSD17B13 enzyme activity measurement, or threshold HSD17B13 enzyme activity.

Some embodiments include identifying, based on the HSD17B13 enzyme activity of the subject, the subject as likely to benefit from a treatment with an HSD17B13 inhibitor or as not likely to benefit from the treatment. Some embodiments include identifying the subject as likely to benefit from the treatment with the HSD17B13 inhibitor when the HSD17B13 enzyme activity of the subject is greater than the control HSD17B13 enzyme activity measurement, baseline HSD17B13 enzyme activity measurement, or threshold HSD17B13 enzyme activity. In some embodiments, the HSD17B13 enzyme activity of the subject is about the same or lower than the control HSD17B13 enzyme activity measurement, baseline HSD17B13 enzyme activity measurement, or threshold HSD17B13 enzyme activity. Some embodiments include identifying the subject as unlikely to benefit from the treatment with the HSD17B13 inhibitor when the HSD17B13 enzyme activity of the subject is about the same or lower than the control HSD17B13 enzyme activity measurement, baseline HSD17B13 enzyme activity measurement, or threshold HSD17B13 enzyme activity.

Some embodiments include identifying, based on the HSD17B13 enzyme activity measurement, a risk that the subject has or will develop a liver disease. Some embodiments include identifying the subject as likely to develop the liver disease when the HSD17B13 enzyme activity of the subject is greater than the control HSD17B13 enzyme activity measurement, baseline HSD17B13 enzyme activity measurement, or threshold HSD17B13 enzyme activity. Examples of liver diseases include liver inflammation, liver fibrosis, cholestasis, a gall bladder disease, a biliary tree disease, alcoholic liver disease, or non-alcoholic steatohepatitis.

In some embodiments, measuring the amount of the reaction product or second amount of the HSD17B13 substrate comprises performing liquid chromatography. In some embodiments, measuring the amount of the reaction product or second amount of the HSD17B13 substrate comprises performing high-performance liquid chromatography. In some embodiments, measuring the amount of the reaction product or second amount of the HSD17B13 substrate comprises mass spectrometry. In some embodiments, measuring the amount of the reaction product or second amount of the HSD17B13 substrate comprises performing liquid chromatography, high-performance liquid chromatography or mass spectrometry. Some embodiments include liquid chromatography coupled to mass spectrometry (e.g. LC/MS-MS). Any HSD17B13 substrates or reaction products described herein may be measured using one or more of these methods, among others.

Some embodiments include a sample such as a patient sample, or a sample from a subject. In some embodiments, the sample comprises a blood sample, a plasma sample or a serum sample. In some embodiments, the sample comprises a blood sample. In some embodiments, the sample comprises a plasma sample. In some embodiments, the sample comprises a serum sample.

In some embodiments, the subject is a mammal. In some embodiments, the subject is a human. In some embodiments, the subject has a liver disease. In some embodiments, the subject is at risk of developing a liver disease. For example, the subject may have an overactive HSD17B13 genotype that results in increased expression of an HSD17B13 gene product.

### In Vitro HSD17B13 Activity and Related Methods

Disclosed herein, in some embodiments, are methods of determining an HSD17B13 enzyme activity in vitro. In the enzyme activity assay, an HSD17B13 substrate or substrate precursor may be used to determine the amount or activity of HSD17B13 in the sample. Some embodiments include contacting a sample with an initial amount of an HSD17B13 substrate described herein. In some embodiments, the contact produces a reaction product from the HSD17B13 substrate or a second amount of the HSD17B13 substrate. Some embodiments include determining a presence or amount of the reaction product or the second amount of the substrate. Some embodiments include a method for determining an HSD17B13 enzyme activity in vitro, comprising: contacting a sample with an initial amount of an HSD17B13 substrate comprising LTB3, resolvin D1, resolvin E1, protectin DX, maresin 1, 12-HETE, 15-HETE, 13-HODE, 15-HEDE, methyl malonyl coenzyme A or lauryl coenzyme A, or a salt thereof, wherein the contact produces a reaction product from the HSD17B13 substrate or a second amount of the HSD17B13 substrate; and determining a presence or amount of the reaction product or the second amount of the substrate. Some embodiments include a method for determining an HSD17B13 enzyme activity in vitro, comprising: contacting a sample with an initial amount of an HSD17B13 substrate comprising LTB3, resolvin D1, resolvin E1, protectin DX, maresin 1, 12-HETE, 13-HODE, 15-HEDE, methyl malonyl coenzyme A or lauryl coenzyme A, or a salt thereof, wherein the contact produces a reaction product from the HSD17B13 substrate or a second amount of the HSD17B13 substrate; and determining a presence or amount of the reaction product or the second amount of the substrate. Where a substrate is used in the in vivo and in vitro methods disclosed herein, a substrate precursor may be used instead, and the reaction products may be measured. In addition, a substrate precursor measurement may also be obtained as part of the assay. In some embodiments, NADH is measured as an output for the assay. For example, a reaction product may include NADH.

Some embodiments include determining a presence or amount of an amount of a reaction product comprising an oxidized HSD17B13 substrate described herein, in a sample that has been contacted with the HSD17B13 substrate. Some embodiments include a method for determining an HSD17B13 enzyme activity, comprising: determining a presence or amount of an amount of a reaction product comprising an oxidized HSD17B13 substrate comprising LTB3, resolvin D1, resolvin E1, protectin DX, maresin 1, 12-HETE, 15-HETE, 13-HODE, 15-HEDE, methyl malonyl coenzyme A or lauryl coenzyme A, or a salt thereof, in a sample that has been contacted with the HSD17B13 substrate.

Some embodiments include a method for determining an HSD17B13 enzyme activity, comprising: determining a presence or amount of an amount of a reaction product comprising an oxidized HSD17B13 substrate comprising LTB3, resolvin D1, resolvin E1, protectin DX, maresin 1, 12-HETE, 15-HETE, 13-HODE, 15-HEDE, methyl malonyl coenzyme A or lauryl coenzyme A, or a salt thereof, in a sample that has been contacted with the HSD17B13 substrate. Some embodiments include a method for determining an HSD17B13 enzyme activity, comprising: determining a presence or amount of an amount of a reaction product comprising an oxidized HSD17B13 substrate comprising LTB3, resolvin D1, resolvin E1, protectin DX, maresin 1, 12-HETE, 13-HODE, 15-HEDE, methyl malonyl coenzyme A or lauryl coenzyme A, or a salt thereof, in a sample that has been contacted with the HSD17B13 substrate.

Some embodiments determining a presence or amount of an amount of a reaction product of a contact of HSD17B13 with an HSD17B13 substrate, in a sample that has been contacted with an HSD17B13 substrate comprising an unoxidized form of the reaction product. Some embodiments include a method for determining an HSD17B13 enzyme activity, comprising: determining a presence or amount of an amount of a reaction product of a contact of HSD17B13 with an HSD17B13 substrate, in a sample that has been contacted with an HSD17B13 substrate comprising an unoxidized form of the reaction product.

Some embodiments include contacting a sample with an HSD17B13 substrate. In some embodiments, the sample is contacted with the HSD17B13 substrate for a length of time (e.g. 15 s, 30 s, 45 s, 1 min, 2 min, 3 min, 4 min, 5 min, 6 min 7 min, 8 min, 9 min, 10 min, 15 min, 30 min 45 min, 1 hr, 2 hrs, 3 hrs, 4 hrs, 5 hrs, 6 hrs, 7 hrs, 8 hrs, 9 hrs, 10 hrs, 11 hrs, 12 hrs, 13 hrs, 14, hrs, 15 hrs, 16 hrs, 17 hrs, 18 hrs, 19 hrs, 20 hrs, 21 hrs, 22 hrs, 23 hrs, 24 hrs, or longer). In some embodiments, the sample is contacted with the HSD17B13 substrate in a buffered solution.

Some embodiments include determining an HSD17B13 enzyme activity or a specific HSD17B13 enzyme activity based on the presence or amount of the reaction product or second amount of the substrate. In some embodiments, the sample comprises a presence or amount of HSD17B13 enzyme activity. In some embodiments, the presence or amount of the reaction product or the second amount of the substrate corresponds to the presence or amount of HSD17B13 enzyme activity in the sample. Some embodiments include determining the presence or amount of HSD17B13 enzyme activity based on the presence or amount of the reaction product or second amount of the substrate. In some embodiments, determining a presence or amount of the reaction product comprises measuring an amount of the reaction product or the second amount of the substrate. Some embodiments include measuring the amount of the reaction product or the second amount of the substrate comprises identifying an area under a curve of the amount of the reaction products or the second amount of the substrate formed over time. In some embodiments, measuring the amount of the reaction product comprises comparing the amount of the reaction product or second amount of the substrate produced in the sample to an amount of the reaction product or second amount of the substrate produced in a control sample. Some embodiments include determining the presence or amount of HSD17B13 enzyme activity based on the presence or amount of the reaction product compared to the second amount of the substrate. Some embodiments include normalizing the determined amount of HSD17B13 enzyme activity using a weight of the sample, a volume of the sample, or an amount of one or more control substances (e.g. HSD17B13, total protein or a housekeeping protein) in the sample to obtain a specific HSD17B13 activity.

In some embodiments, the sample is contacted with the HSD17B13 substrate in vitro. In some embodiments, measuring the amount of the reaction product comprises performing liquid chromatography, high-performance liquid chromatography or mass spectrometry. Some embodiments include liquid chromatography coupled to mass spectrometry (e.g. LC/MS-MS).

In some embodiments, the sample comprises a biological sample. In some embodiments, the biological sample is from a mammal. In some embodiments, the biological sample is from a human. In some embodiments, the sample comprises a blood sample, a plasma sample or a serum sample. In some embodiments, the sample is a plasma sample. In some embodiments, the sample comprises cells or a cell lysate. In some embodiments, the sample comprises a tissue or tissue homogenate.

Disclosed herein, in some embodiments, are methods of treating a patient based on an in vitro HSD17B13 activity measurement. In some embodiments, the in vitro HSD17B13 activity measurement is obtained using a method described herein. Some embodiments include obtaining or receiving an HSD17B13 enzyme activity measurement obtained by contacting a sample from a patient with an HSD17B13 substrate described herein. Some embodiments include determining a presence or amount of a reaction product of HSD17B13 with the HSD17B13 substrate. Some embodiments include administering a 17β-Hydroxysteroid dehydrogenase type 13 (HSD17B13) inhibitor based on the HSD17B13 enzyme activity measurement. Some embodiments include a method of treating a patient, comprising: obtaining or receiving an HSD17B13 enzyme activity measurement obtained by contacting a sample from a patient with an HSD17B13 substrate comprising LTB3, resolvin D1, resolvin E1, protectin DX, maresin 1, 12-HETE, 15-HETE, 13-HODE, 15-HEDE, methyl malonyl coenzyme A or lauryl coenzyme A, or a salt thereof, and determining a presence or amount of a reaction product of HSD17B13 with the HSD17B13 substrate; and administering a 17P-Hydroxysteroid dehydrogenase type 13 (HSD17B13) inhibitor based on the HSD17B13 enzyme activity measurement. Some embodiments include a method of treating a patient, comprising: obtaining or receiving an HSD17B13 enzyme activity measurement obtained by contacting a sample from a patient with an HSD17B13 substrate comprising LTB3, resolvin D1, resolvin E1, protectin DX, maresin 1, 12-HETE, 13-HODE, 15-HEDE, methyl malonyl coenzyme A or lauryl coenzyme A, or a salt thereof, and determining a presence or amount of a reaction product of HSD17B13 with the HSD17B13 substrate; and administering a 17β-Hydroxysteroid dehydrogenase type 13 (HSD17B13) inhibitor based on the HSD17B13 enzyme activity measurement.

Some embodiments include obtaining or receiving an HSD17B13 enzyme activity measurement obtained by contacting a sample from a patient with an HSD17B13 substrate described herein. Some embodiments include determining a presence or amount of a reaction product. Some embodiments include identifying, based on the HSD17B13 enzyme activity measurement, the patient as likely to benefit from a treatment with an HSD17B13 inhibitor or as not likely to benefit from the treatment. Some embodiments include a method for identifying a patient likely to benefit from treatment with an HSD17B13 inhibitor, comprising: obtaining or receiving an HSD17B13 enzyme activity measurement obtained by contacting a sample from a patient with an HSD17B13 substrate comprisingLTB3, resolvin D1, resolvin E1, protectin DX, maresin 1, 12-HETE, 15-HETE, 13-HODE, 15-HEDE, methyl malonyl coenzyme A or lauryl coenzyme A, or a salt thereof, and determining a presence or amount of a reaction product; and identifying, based on the HSD17B13 enzyme activity measurement, the patient as likely to benefit from a treatment with an HSD17B13 inhibitor or as not likely to benefit from the treatment. Some embodiments include a method for identifying a patient likely to benefit from treatment with an HSD17B13 inhibitor, comprising: obtaining or receiving an HSD17B13 enzyme activity measurement obtained by contacting a sample from a patient with an HSD17B13 substrate comprisingLTB3, resolvin D1, resolvin E1, protectin DX, maresin 1, 12-HETE, 13-HODE, 15-HEDE, methyl malonyl coenzyme A or lauryl coenzyme A, or a salt thereof, and determining a presence or amount of a reaction product; and identifying, based on the HSD17B13 enzyme activity measurement, the patient as likely to benefit from a treatment with an HSD17B13 inhibitor or as not likely to benefit from the treatment.

Some embodiments include comparing the HSD17B13 enzyme activity measurement to a control HSD17B13 enzyme activity measurement, a baseline HSD17B13 enzyme activity measurement or a threshold. In some embodiments, the control HSD17B13 enzyme activity measurement is obtained from a general patient population. In some embodiments, the HSD17B13 enzyme activity measurement is greater than the control HSD17B13 enzyme activity measurement, baseline HSD17B13 enzyme activity measurement or threshold.

Some embodiments include the patient as likely to benefit from a treatment with an HSD17B13 inhibitor or as not likely to benefit from the treatment. Some embodiments include identifying the patient as likely to benefit from the treatment with the HSD17B13 inhibitor when the HSD17B13 enzyme activity measurement is greater than the control HSD17B13 enzyme activity measurement, baseline HSD17B13 enzyme activity measurement or threshold. The HSD17B13 inhibitor may be an HSD17B13 inhibitor described herein. Some embodiments include providing the treatment with the HSD17B13 inhibitor to the patient.

In some embodiments, the HSD17B13 enzyme activity measurement is about the same or lower than the control HSD17B13 enzyme activity measurement, baseline HSD17B13 enzyme activity measurement or threshold. Some embodiments include identifying the patient as unlikely to benefit from the treatment with the HSD17B13 inhibitor when the HSD17B13 enzyme activity measurement is about the same or lower than the control HSD17B13 enzyme activity measurement, baseline HSD17B13 enzyme activity measurement or threshold. Some embodiments include identifying, based on the HSD17B13 enzyme activity measurement, a risk that the patient has or will develop a liver disease. Some embodiments include identifying the patient as likely to develop the liver disease when the HSD17B13 enzyme activity measurement is greater than the control HSD17B13 enzyme activity measurement, baseline HSD17B13 enzyme activity measurement or threshold. The liver disease may be a liver disease described herein.

Some embodiments include obtaining or receiving an HSD17B13 enzyme activity measurement obtained by contacting a sample from a patient with an HSD17B13 substrate described herein. Some embodiments include determining a presence or amount of a reaction product of the HSD17B13 substrate. Some embodiments include identifying, based on the HSD17B13 enzyme activity measurement, a risk that the patient has or will develop a liver disease. Some embodiments include a method for identifying a risk that a patient has or will develop a liver disease, comprising: obtaining or receiving an HSD17B13 enzyme activity measurement obtained by contacting a sample from a patient with an HSD17B13 substrate comprising LTB3, resolvin D1, resolvin E1, protectin DX, maresin 1, 12-HETE, 13-HODE, 15-HEDE, methyl malonyl coenzyme A or lauryl coenzyme A, or a salt thereof, and determining a presence or amount of a reaction product; and identifying, based on the HSD17B13 enzyme activity measurement, a risk that the patient has or will develop a liver disease. Some embodiments include a method for identifying a risk that a patient has or will develop a liver disease, comprising: obtaining or receiving an HSD17B13 enzyme activity measurement obtained by contacting a sample from a patient with an HSD17B13 substrate comprising LTB3, resolvin D1, resolvin E1, protectin DX, maresin 1, 12-HETE, 15-HETE, 13-HODE, 15-HEDE, methyl malonyl coenzyme A or lauryl coenzyme A, or a salt thereof, and determining a presence or amount of a reaction product; and identifying, based on the HSD17B13 enzyme activity measurement, a risk that the patient has or will develop a liver disease. Some embodiments include identifying the patient as unlikely to develop the liver disease when the HSD17B13 enzyme activity measurement is about the same or lower than the control HSD17B13 enzyme activity measurement, baseline HSD17B13 enzyme activity measurement or threshold.

Some embodiments include obtaining or receiving an HSD17B13 enzyme activity measurement in a sample from a patient receiving an HSD17B13 inhibitor treatment, the HSD17B13 enzyme activity measurement having been obtained by contacting a sample from a patient with an HSD17B13 substrate described herein, and determining a presence or amount of a reaction product. Some embodiments include evaluating the HSD17B13 inhibitor treatment of the patient based on the HSD17B13 enzyme activity measurement. Some embodiments include a method for evaluating an HSD17B13 inhibitor treatment of a patient, comprising: obtaining or receiving an HSD17B13 enzyme activity measurement in a sample from a patient receiving an HSD17B13 inhibitor treatment, the HSD17B13 enzyme activity measurement having been obtained by contacting a sample from a patient with an HSD17B13 substrate comprising LTB3, resolvin D1, resolvin E1, protectin DX, maresin 1, 12-HETE, 15-HETE, 13-HODE, 15-HEDE, methyl malonyl coenzyme A or lauryl coenzyme A, or a salt thereof, and determining a presence or amount of a reaction product; and evaluating the HSD17B13 inhibitor treatment of the patient based on the HSD17B13 enzyme activity measurement. Some embodiments include a method for evaluating an HSD17B13 inhibitor treatment of a patient, comprising: obtaining or receiving an HSD17B13 enzyme activity measurement in a sample from a patient receiving an HSD17B13 inhibitor treatment, the HSD17B13 enzyme activity measurement having been obtained by contacting a sample from a patient with an HSD17B13 substrate comprising LTB3, resolvin D1, resolvin E1, protectin DX, maresin 1, 12-HETE, 13-HODE, 15-HEDE, methyl malonyl coenzyme A or lauryl coenzyme A, or a salt thereof, and determining a presence or amount of a reaction product; and evaluating the HSD17B13 inhibitor treatment of the patient based on the HSD17B13 enzyme activity measurement.

Some embodiments include comparing the HSD17B13 enzyme activity measurement to a baseline HSD17B13 enzyme activity measurement. Some embodiments include determining whether the HSD17B13 enzyme activity measurement is significantly different from the baseline HSD17B13 enzyme activity measurement. In some embodiments, the HSD17B13 enzyme activity measurement is significantly different from the baseline HSD17B13 enzyme activity measurement when the HSD17B13 enzyme activity measurement is at least 10%, at least 25%, or at least 50% different from the baseline measurement. In some embodiments, the HSD17B13 enzyme activity measurement is significantly lower than the baseline HSD17B13 enzyme activity measurement. Some embodiments include determining that the HSD17B13 inhibitor treatment of the patient is successful when the HSD17B13 enzyme activity measurement is significantly lower than the baseline HSD17B13 enzyme activity measurement. In some embodiments, the HSD17B13 enzyme activity measurement is about the same or higher than the baseline HSD17B13 enzyme activity measurement. Some embodiments include determining that the HSD17B13 inhibitor treatment of the patient is unsuccessful when the HSD17B13 enzyme activity measurement is about the same or higher than the baseline HSD17B13 enzyme activity measurement.

Some embodiments include increasing a dose or amount of HSD17B13 inhibitor in a subsequent HSD17B13 inhibitor treatment, or recommending an increase in the dose or amount of a subsequent HSD17B13 inhibitor treatment, based on the evaluation of the HSD17B13 inhibitor treatment. Some embodiments include increasing a dose or amount of HSD17B13 inhibitor in a subsequent HSD17B13 inhibitor treatment, or recommending a decrease in the dose or amount of a subsequent HSD17B13 inhibitor treatment, when the HSD17B13 inhibitor treatment of the patient is successful or unsuccessful. Some embodiments include decreasing a dose or amount of HSD17B13 inhibitor in a subsequent HSD17B13 inhibitor treatment, or recommending a decrease in the dose or amount of a subsequent HSD17B13 inhibitor treatment, based on the evaluation of the HSD17B13 inhibitor treatment. Some embodiments include decreasing a dose or amount of HSD17B13 inhibitor in a subsequent HSD17B13 inhibitor treatment, or recommending a decrease in the dose or amount of a subsequent HSD17B13 inhibitor treatment, when the HSD17B13 inhibitor treatment of the patient is successful or unsuccessful.

In some embodiments of the methods described herein, the patient is a mammal. In some embodiments, the patient is a human.

### HSD17B13 Inhibitors and Compositions Including HSD17B13 Inhibitors

Described herein, in some embodiments, are HSD17B13 inhibitors or compositions including HSD17B13 inhibitors. The HSD17B13 inhibitors may be used in a method described herein. For example, an HSD17B13 inhibitor may be administered or evaluated in accordance with a method described herein. An HSD17B13 inhibitor may be administered in a method with an HSD17B13 substrate to determine a correct dose to provide to a subject. The HSD17B13 inhibitors may be included in a kit.

Described herein, in some embodiments, are HSD17B13 inhibitors. In some embodiments, the HSD17B13 inhibitor reduces HSD17B13 activity by at least 10%. In some embodiments, the HSD17B13 inhibitor reduces HSD17B13 expression by at least 10%. The reduced activity or expression may be compared to a baseline measurement. The baseline measurement may be measured in a first patient sample, and the reduced activity or expression may be measured in a second patient sample.

Described herein, in some embodiments, are HSD17B13 inhibitors. In some embodiments, the HSD17B13 inhibitor comprises a small molecule, a polypeptide, or an oligonucleotide. In some embodiments, the HSD17B13 inhibitor comprises a small molecule. In some embodiments, the HSD17B13 inhibitor comprises a polypeptide. In some embodiments, the HSD17B13 inhibitor comprises an oligonucleotide.

In some embodiments, the HSD17B13 inhibitor comprises an estradiol mimetic, propyl pyrazole triole, an anti-HSD17B13 antibody or antibody fragment, an oligonucleotide directed against an HSD17B13 encoding oligonucleotide, an antisense oligonucleotide, or an siRNA. In some embodiments, the HSD17B13 inhibitor comprises an estradiol mimetic. In some embodiments, the HSD17B13 inhibitor comprises propyl pyrazole triole. In some embodiments, the HSD17B13 inhibitor comprises an anti-HSD17B13 antibody or antibody fragment. In some embodiments, the HSD17B13 inhibitor comprises an oligonucleotide directed against an HSD17B13 encoding oligonucleotide. For example, the HSD17B13 inhibitor may include a nucleic acid sequence that comprises or is complementary to a nucleic acid sequence encoding HSD17B13. In some embodiments, the HSD17B13 inhibitor comprises an antisense oligonucleotide. In some embodiments, the HSD17B13 inhibitor comprises or an siRNA. In some embodiments, the HSD17B13 inhibitor comprises ARO-HSD. In some embodiments, the HSD17B13 inhibitor comprises ALN-HSD.

Described herein, in some embodiments, are HSD17B13 inhibitors comprising antisense oligonucleotides (ASOs). In some embodiments, the composition comprises an oligonucleotide that inhibits the expression of HSD17B13, wherein the oligonucleotide comprises an antisense oligonucleotide (ASO). In some embodiments, the ASO is 12-30 nucleosides in length. In some embodiments, the ASO is 14-30 nucleosides in length. In some embodiments, the ASO is at least about 10, 11, 12, 13, 14, 15, 16, 17, 18,19, 20, 21, 22, 23, 24, 25, 26, 27, 28, 29, or 30 nucleosides in length, or a range defined by any of the two aforementioned numbers. In some embodiments, the ASO is 15-25 nucleosides in length. In some embodiments, the ASO is 20 nucleosides in length.

Described herein, in some embodiments, are HSD17B13 inhibitors comprising ASOs. In some embodiments, the composition comprises an oligonucleotide that inhibits the expression of HSD17B13, wherein the oligonucleotide comprises an ASO about 12-30 nucleosides in length and comprising a nucleoside sequence comprising about 12-30 contiguous nucleosides of a full-length human HSD17B13 mRNA sequence; wherein (i) the oligonucleotide comprises a modification comprising a modified nucleoside and/or a modified internucleoside linkage, and/or (ii) the composition comprises a pharmaceutically acceptable carrier.

Described herein, in some embodiments, are HSD17B13 inhibitors comprising small interfering RNAs (siRNAs). In some embodiments, the composition comprises an oligonucleotide that targets HSD17B13, wherein the oligonucleotide comprises an siRNA. In some embodiments, the composition comprises an oligonucleotide that targets HSD17B13, wherein the oligonucleotide comprises an siRNA comprising a sense strand and an antisense strand.

Described herein, in some embodiments, are HSD17B13 inhibitors comprising siRNA. In some embodiments, the composition comprises an oligonucleotide that inhibits the expression of HSD17B13, wherein the oligonucleotide comprises an siRNA comprising a sense strand and an antisense strand, wherein the sense strand is 14-30 nucleosides in length. In some embodiments, the composition comprises a sense strange that is at least about 10, 11, 12, 13, 14, 15, 16, 17, 18,19, 20, 21, 22, 23, 24, 25, 26, 27, 28, 29, or 30 nucleosides in length, or a range defined by any of the two aforementioned numbers. In some embodiments, the composition comprises an antisense strand is 14-30 nucleosides in length. In some embodiments, the composition comprises an antisense strange that is at least about 10, 11, 12, 13, 14, 15, 15, 17, 18, 19, 20, 21, 22, 23, 24, 25, 26, 27, 28, 29, or 30 nucleosides in length, or a range defined by any of the two aforementioned numbers.

In some embodiments, the HSD17B13 inhibitor is:

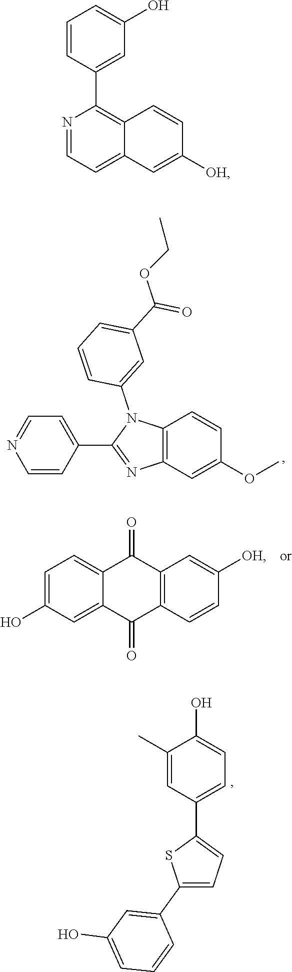

or a pharmaceutically acceptable salt or derivative thereof.

In some embodiments, the HSD17B13 inhibitor is:

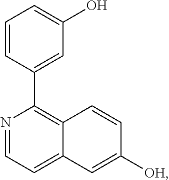 [SMILES: Oc1cccc(-c2nccc3cc(O)ccc23)c1]

or a pharmaceutically acceptable salt or derivative thereof. In some embodiments, the HSD17B13 inhibitor is:

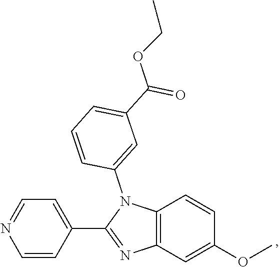 [SMILES: CCOC(=O)c1cccc(-n2c(-c3ccncc3)nc3cc(OC)ccc32)c1]

or a pharmaceutically acceptable salt or derivative thereof.

In some embodiments, the HSD17B13 inhibitor is:

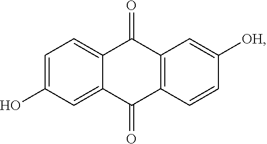 [SMILES: O=C1c2ccc(O)cc2C(=O)c2ccc(O)cc21]

or a pharmaceutically acceptable salt or derivative thereof.

In some embodiments, the HSD17B13 inhibitor is:

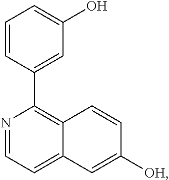 [SMILES: Oc1cccc(-c2nccc3cc(O)ccc23)c1]

or a pharmaceutically acceptable salt or derivative thereof. In some embodiments, the HSD17B13 inhibitor is

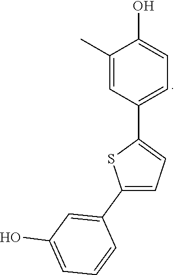 [SMILES: Cc1cc(-c2ccc(-c3cccc(O)c3)s2)ccc1O]

In some embodiments, the HSD17B13 inhibitor is:

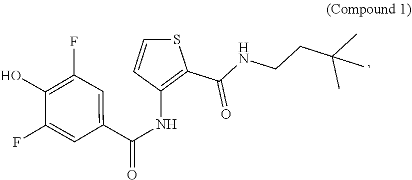 [SMILES: CC(C)(C)CCNC(=O)c1sccc1NC(=O)c1cc(F)c(O)c(F)c1]

or a pharmaceutically acceptable salt or derivative thereof.

Some examples of HSD17B13 inhibitor are included in CN103520724B, WO2001019798, WO2002096873, WO2019183329, WO2020051207, or US20180291422, each of which is incorporated herein by reference.

Described herein, in some embodiments, are liver disease treatments comprising a composition including an HSD17B13 inhibitor. In some embodiments, the composition is a pharmaceutical composition. In some embodiments, the composition is sterile. In some embodiments, the composition further comprises a pharmaceutically acceptable carrier. In some embodiments, the pharmaceutically acceptable carrier comprises water. In some embodiments, the pharmaceutically acceptable carrier comprises a buffer. In some embodiments, the pharmaceutically acceptable carrier comprises a saline solution. In some embodiments, the pharmaceutically acceptable carrier comprises water, a buffer, or a saline solution. In some embodiments, the composition comprises a liposome. In some embodiments, the pharmaceutically acceptable carrier comprises liposomes, lipids, nanoparticles, proteins, protein-antibody complexes, peptides, cellulose, nanogel, or a combination thereof.

### Kits

Disclosed herein, in some embodiments, are kits. Some embodiments include an HSD17B13 substrate. Some embodiments include an HSD17B13 substrate precursor. In some embodiments, the HSD17B13 substrate is labeled. In some embodiments, the HSD17B13 substrate is radiolabeled. In some embodiments, the radiolabeled HSD17B13 substrate comprises 13C. In some embodiments, the radiolabeled HSD17B13 substrate comprises 2H. In some embodiments, the radiolabeled HSD17B13 substrate comprises 13C or 2H. In some embodiments, the radiolabeled HSD17B13 substrate comprises 13C and 2H.

Some embodiments include a reagent for measuring the HSD17B13 substrate. Some embodiments include a reagent for specifically measuring the HSD17B13 substrate. Some embodiments include a reagent for specifically measuring the labeled HSD17B13 substrate. Some embodiments include an oxidized form of an HSD17B13 substrate. In some embodiments, the oxidized form of the HSD17B13 substrate is labeled. Some embodiments include a reagent for measuring an oxidized form of the HSD17B13 substrate. Some embodiments include a reagent for specifically measuring an oxidized form of the HSD17B13 substrate. In some embodiments, the HSD17B13 substrate comprises a primary alcohol, and the reaction product comprises an aldehyde. In some embodiments, the HSD17B13 substrate comprises a secondary alcohol, and the reaction product comprises a ketone.

Some embodiments include reagents for measuring any or all of the following HSD17B13 substrates. LTB3, resolvin D1, resolvin E1, protectin DX, maresin 1, 12-HETE, 15-HETE, 13-HODE, 15-HEDE, methyl malonyl coenzyme A or lauryl coenzyme A, or a salt thereof. Some embodiments include reagents for measuring one or more oxidized forms of any or all of the following HSD17B13 substrates. LTB3, resolvin D1, resolvin E1, protectin DX, maresin 1, 12-HETE, 15-HETE, 13-HODE, 15-HEDE, methyl malonyl coenzyme A or lauryl coenzyme A, or a salt thereof. Some embodiments include an HSD17B13 substrate comprising LTB3, resolvin D1, resolvin E1, protectin DX, maresin 1, 12-HETE, 15-HETE, 13-HODE, 15-HEDE, methyl malonyl coenzyme A or lauryl coenzyme A, or a salt thereof; and a reagent for measuring a reaction product of the HSD17B13 substrate, or a reagent for measuring the HSD17B 13 substrate.

Some embodiments include reagents for measuring any or all of the following HSD17B13 substrates. LTB3, resolvin D1, resolvin E1, protectin DX, maresin 1, 12-HETE, 13-HODE, 15-HEDE, methyl malonyl coenzyme A or lauryl coenzyme A, or a salt thereof. Some embodiments include reagents for measuring one or more oxidized forms of any or all of the following HSD17B13 substrates. LTB3, resolvin D1, resolvin E1, protectin DX, maresin 1, 12-HETE, 13-HODE, 15-HEDE, methyl malonyl coenzyme A or lauryl coenzyme A, or a salt thereof. Some embodiments include an HSD17B13 substrate comprising LTB3, resolvin D1, resolvin E1, protectin DX, maresin 1, 12-HETE, 13-HODE, 15-HEDE, methyl malonyl coenzyme A or lauryl coenzyme A, or a salt thereof; and a reagent for measuring a reaction product of the HSD17B13 substrate, or a reagent for measuring the HSD17B13 substrate.

Some embodiments include a solution containing a known concentration of HSD17B13. Some embodiments include instructions for using the reagent in an HSD17B13 activity assay. In some embodiments, the kit is packaged.

### Modified Proteins and Ligand-Protein Complexes

Disclosed herein, in some embodiments, are in-vivo modified proteins comprising an HSD17B13 enzyme bound to a ligand, wherein the ligand comprises a labeled HSD17B13 substrate comprising LTB3, resolvin D1, resolvin E1, protectin DX, maresin 1, 12-HETE, 15-HETE, 13-HODE, 15-HEDE, methyl malonyl coenzyme A or lauryl coenzyme A, or a salt thereof, or wherein the ligand comprises a labeled reaction product of the HSD17B13 substrate. Disclosed herein, in some embodiments, are in-vivo modified proteins comprising an HSD17B13 enzyme bound to a ligand, wherein the ligand comprises a labeled HSD17B13 substrate comprising LTB3, resolvin D1, resolvin E1, protectin DX, maresin 1, 12-HETE, 13-HODE, 15-HEDE, methyl malonyl coenzyme A or lauryl coenzyme A, or a salt thereof, or wherein the ligand comprises a labeled reaction product of the HSD17B13 substrate. In some embodiments, the contact comprises a non-covalent bond between the HSD17B13 enzyme and the ligand. In some embodiments, the non-covalent bond comprises a hydrogen bond, an ionic bond, a van der Waals interaction, or a hydrophobic bond. In some embodiments, the HSD17B13 substrate is radiolabeled. In some embodiments, the radiolabeled HSD17B13 substrate comprises 13C or 2H. In some embodiments, the reaction product comprises an oxidized form of the HSD17B13 substrate. In some embodiments, the HSD17B13 substrate comprises a primary alcohol, and the reaction product comprises an aldehyde. In some embodiments, the HSD17B13 substrate comprises a secondary alcohol, and the reaction product comprises a ketone.

Disclosed herein, in some embodiments, are ligand-HSD17B13 complexes formed by binding an HSD17B13 enzyme to a ligand, the ligand comprising a labeled HSD17B13 substrate comprising LTB3, resolvin D1, resolvin E1, protectin DX, maresin 1, 12-HETE, 15-HETE, 13-HODE, 15-HEDE, methyl malonyl coenzyme A or lauryl coenzyme A, or a salt thereof, or the ligand comprising a labeled reaction product of the HSD17B13 substrate. Disclosed herein, in some embodiments, are ligand-HSD17B13 complexes formed by binding an HSD17B13 enzyme to a ligand, the ligand comprising a labeled HSD17B13 substrate comprising LTB3, resolvin D1, resolvin E1, protectin DX, maresin 1, 12-HETE, 13-HODE, 15-HEDE, methyl malonyl coenzyme A or lauryl coenzyme A, or a salt thereof, or the ligand comprising a labeled reaction product of the HSD17B13 substrate. In some embodiments, the contact comprises a non-covalent bond between the HSD17B13 enzyme and the ligand. In some embodiments, the non-covalent bond comprises a hydrogen bond, an ionic bond, a van der Waals interaction, or a hydrophobic bond. In some embodiments, the HSD17B13 substrate is radiolabeled. In some embodiments, the radiolabeled HSD17B13 substrate comprises 13C or 2H. In some embodiments, the reaction product comprises an oxidized form of the HSD17B13 substrate. In some embodiments, the HSD17B13 substrate comprises a primary alcohol, and the reaction product comprises an aldehyde. In some embodiments, the HSD17B13 substrate comprises a secondary alcohol, and the reaction product comprises a ketone.

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

The terms “subject,” “individual,” or “patient” are often used interchangeably herein. A “subject” can be a biological entity containing expressed genetic materials. The biological entity can be an animal. The subject can be tissues, cells and their progeny of a biological entity obtained in vivo or cultured in vitro. The subject can be a mammal. The mammal can be a human. The subject may be diagnosed or suspected of being at high risk for a disease. In some cases, the subject is not necessarily diagnosed or suspected of being at high risk for the disease. A patient described herein, may in some embodiments, be a non-human subject. In some embodiments, the patient is a human. In some embodiments, the patient is female. In some embodiments, the patient is male.

As used herein, the term “about” a number refers to that number plus or minus 15% of that number. The term “about” a range refers to that range minus 15% of its lowest value and plus 15% of its greatest value.

As used herein, the terms “treatment” or “treating” are used in reference to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

The section headings used herein are for organizational purposes only and are not to be construed as limiting the subject matter described.

### EMBODIMENTS

Embodiment 1. A method of treatment, comprising:
administering to a subject a first amount of a 17β-Hydroxysteroid dehydrogenase type 13 (HSD17B13) substrate; and
determining an amount of a reaction product of the HSD17B13 substrate in a sample obtained from the subject, or determining a second amount of the HSD17B13 substrate in a sample obtained from the subject after administering the first amount of the HSD17B13 substrate to the subject.

Embodiment 2. The method of embodiment 1, wherein the HSD17B13 substrate comprises LTB3, resolvin D1, resolvin E1, protectin DX, maresin 1, 12-HETE, 13-HODE, 15-HEDE, methyl malonyl coenzyme A, or lauryl coenzyme A, or a salt thereof.

Embodiment 3. The method of embodiment 1 or 2, wherein the reaction product comprises an oxidized form of the HSD17B13 substrate.

Embodiment 4. The method of any one of embodiments 1-3, wherein the HSD17B13 substrate comprises a primary alcohol, and the reaction product comprises an aldehyde.

Embodiment 5. The method of any one of embodiments 1-4, wherein the HSD17B13 substrate comprises a secondary alcohol, and the reaction product comprises a ketone.

Embodiment 6. The method of any one of embodiments 1-5, wherein the sample is obtained from the subject about 1 min, about 5 min, about 10 min, about 15 min, about 20 min, about 25 min, about 30 min, about 35 min, about 40 min, about 45 min, about 50 min, about 55 min, about 1 hr, about 2 hr, about 4 hr, about 6 hr, about 8 hr, about 10 hr, about 12 hr, about 14 hr, about 16 hr, about 18 hr, about 20 hr, about 22 hr, about 24 hr, or longer, after administering the first amount of the HSD17B13 substrate to the subject.

Embodiment 7. The method of any one of embodiments 1-6, further comprising administering or coadministering a first amount of an HSD17B13 inhibitor to the subject with the administration of the first amount of the HSD17B13 substrate.

Embodiment 8. The method of any one of embodiments 1-7, further comprising determining an HSD17B13 enzyme activity based on the amount of the reaction product or second amount of the HSD17B13 substrate in the sample.

Embodiment 9. The method of any one of embodiments 1-8, wherein the administration of the first amount of the HSD17B13 substrate is intravenous or oral.

Embodiment 10. The method of any one of embodiments 1-9, wherein the first amount of the HSD17B13 substrate is 10-1000 μg of the HSD17B13 substrate.

Embodiment 11. The method of any one of embodiments 1-10, wherein the first amount of the HSD17B13 substrate is about 100 μg of the HSD17B13 substrate.

Embodiment 12. The method of any one of embodiments 1-11, wherein the HSD17B13 substrate and the HSD17B13 reaction product are labeled.

Embodiment 13. The method of any one of embodiments 1-12, wherein the HSD17B13 substrate and the HSD17B13 reaction product are radiolabeled.

Embodiment 14. The method of embodiment 13, wherein the radiolabeled HSD17B13 substrate and reaction product comprise 13C or 2H.

Embodiment 15. The method of embodiment 13 or 14, wherein determining the amount of the reaction product comprises measuring the radiolabeled reaction product in the sample.

Embodiment 16. The method of any one of embodiments 13-15, wherein determining the second amount of the HSD17B13 substrate comprises measuring the radiolabeled HSD17B13 substrate in the sample.

Embodiment 17. The method of any one of embodiments 1-16, comprising determining the amount of the reaction product of the HSD17B13 substrate and the second amount of the HSD17B13 substrate in the sample.

Embodiment 18. The method of any one of embodiments 1-17, further comprising determining a second amount of the reaction product of the HSD17B13 substrate in a second sample, or determining a third amount of the HSD17B13 substrate in the second sample, wherein the second sample is obtained from the subject at a second time after administering the first amount of the HSD17B13 substrate to the subject.

Embodiment 19. The method of embodiment 18, wherein the second time comprises about 2 min, about 5 min, about 10 min, about 15 min, about 20 min, about 25 min, about 30 min, about 35 min, about 40 min, about 45 min, about 50 min, about 55 min, about 1 hr, about 2 hr, about 4 hr, about 6 hr, about 8 hr, about 10 hr, about 12 hr, about 14 hr, about 16 hr, about 18 hr, about 20 hr, about 22 hr, about 24 hr, or longer.

Embodiment 20. The method of embodiment 18 or 19, further comprising comparing the second amount of the reaction product to the first amount of the reaction product, or comparing the third amount of the HSD17B13 substrate to the second amount of the HSD17B13 substrate.

Embodiment 21. The method of any one of embodiments 18-20, further comprising generating an area under the curve of the first and second amounts of the reaction product, or generating an area under the curve of the second and third amounts of the HSD17B13 substrate.

Embodiment 22. The method of any one of embodiments 18-21, further comprising comparing a first area under the curve of the first and second amounts of the reaction product, or the second and third amounts of the HSD17B13 substrate, to a second area under the curve of amounts of the reaction product or HSD17B13 substrate, wherein the first area under the curve is generated when the HSD17B13 substrate is coadministered to the subject with an HSD17B13 inhibitor, and wherein the second area under the curve is generated when the HSD17B13 substrate is not coadministered to the subject with the HSD17B13 inhibitor.

Embodiment 23. The method of any one of embodiments 18-21, further comprising comparing a first area under the curve of the first and second amounts of the reaction product, or the second and third amounts of the HSD17B13 substrate, to a second area under the curve of amounts of the reaction product or HSD17B13 substrate, wherein the first area under the curve is generated when the HSD17B13 substrate is coadministered to the subject with a first amount of an HSD17B13 inhibitor, and wherein the second area under the curve is generated when the HSD17B13 substrate is coadministered to the subject with a second amount of the HSD17B13 inhibitor.

Embodiment 24. The method of any one of embodiments 7-23, further comprising comparing the HSD17B13 enzyme activity to a baseline HSD17B13 enzyme activity obtained without coadministering the HSD17B13 inhibitor to the subject with the HSD17B13 substrate.

Embodiment 25. The method of any one of embodiments 8-24, further comprising administering a second amount of the HSD17B13 inhibitor to the subject based on the HSD17B13 enzyme activity.

Embodiment 26. The method of embodiment 24 or 25, further comprising comparing the HSD17B13 enzyme activity to the baseline HSD17B13 enzyme activity, and determining based on the comparison whether to increase, decrease, or not change the second amount of the HSD17B13 inhibitor relative to the first amount of the HSD17B13 inhibitor.

Embodiment 27. The method of embodiment 25 or 26, wherein the second amount of the HSD17B13 inhibitor is greater than the first amount of the HSD17B13 inhibitor.

Embodiment 28. The method of embodiment 25 or 26, wherein the second amount of the HSD17B13 inhibitor is less than the first amount of the HSD17B13 inhibitor.

Embodiment 29. The method of embodiment 25 or 26, wherein the second amount of the HSD17B13 inhibitor is about the same as the first amount of the HSD17B13 inhibitor.

Embodiment 30. The method of any of embodiments 1-29, further comprising administering, with the first amount of the HSD17B13 substrate, a first amount of an HSD17B13 inhibitor, wherein the first amount of the HSD17B13 inhibitor inhibits reaction product formation by about 25%, about 50%, about 75%, about 85%, about 90%, or about 95%.

Embodiment 31. The method of embodiment 30, further comprising determining the amount of the HSD17B13 inhibitor that inhibits reaction product formation by about 25%, about 50%, about 75%, about 85%, about 90%, or about 95%.

Embodiment 32. The method of embodiment 31, further comprising, based on the determined amount of HSD17B13 inhibitor that inhibits reaction product formation by about 25%, about 50%, about 75%, about 85%, about 90%, or about 95%, identifying a dose of the HSD17B13 inhibitor to administer to the subject for treatment.

Embodiment 33. The method of embodiment 32, wherein the dose of the HSD17B13 inhibitor is sufficient to treat a liver disease in the patient.

Embodiment 34. The method of any of embodiments 8-33, further comprising normalizing the HSD17B13 enzyme activity to obtain a specific HSD17B13 enzyme activity of the subject.

Embodiment 35. The method of embodiment 34, wherein the HSD17B13 enzyme activity is normalized to an amount of the sample to obtain the specific HSD17B13 enzyme activity of the subject.

Embodiment 36. The method of any of embodiments 8-35, further comprising comparing the HSD17B13 enzyme activity of the subject to a control HSD17B13 enzyme activity measurement, a baseline HSD17B13 enzyme activity measurement, or a threshold HSD17B13 enzyme activity.

Embodiment 37. The method of embodiment 36, wherein the control HSD17B13 enzyme activity measurement is obtained from a general subject population.

Embodiment 38. The method of embodiment 36 or 37, wherein the HSD17B13 enzyme activity of the subject is greater than the control HSD17B13 enzyme activity measurement, baseline HSD17B13 enzyme activity measurement, or threshold HSD17B13 enzyme activity.

Embodiment 39. The method of any of embodiments 8-38, further comprising identifying, based on the HSD17B13 enzyme activity of the subject, the subject as likely to benefit from a treatment with an HSD17B13 inhibitor or as not likely to benefit from the treatment.

Embodiment 40. The method of any of embodiments 36-39, further comprising identifying the subject as likely to benefit from the treatment with the HSD17B13 inhibitor when the HSD17B13 enzyme activity of the subject is greater than the control HSD17B13 enzyme activity measurement, baseline HSD17B13 enzyme activity measurement, or threshold HSD17B13 enzyme activity.

Embodiment 41. The method of embodiment 36 or 37, wherein the HSD17B13 enzyme activity of the subject is about the same or lower than the control HSD17B13 enzyme activity measurement, baseline HSD17B13 enzyme activity measurement, or threshold HSD17B13 enzyme activity.

Embodiment 42. The method of any of embodiments 36, 47, or 41, further comprising identifying the subject as unlikely to benefit from the treatment with the HSD17B13 inhibitor when the HSD17B13 enzyme activity of the subject is about the same or lower than the control HSD17B13 enzyme activity measurement, baseline HSD17B13 enzyme activity measurement, or threshold HSD17B13 enzyme activity.

Embodiment 43. The method of any of embodiments 36-42, further comprising identifying, based on the HSD17B13 enzyme activity measurement, a risk that the subject has or will develop a liver disease.

Embodiment 44. The method of embodiment 43, further comprising identifying the subject as likely to develop the liver disease when the HSD17B13 enzyme activity of the subject is greater than the control HSD17B13 enzyme activity measurement, baseline HSD17B13 enzyme activity measurement, or threshold HSD17B13 enzyme activity.

Embodiment 45. The method of any of embodiments 33, 43, or 44, wherein the liver disease comprises liver inflammation, liver fibrosis, cholestasis, a gall bladder disease, a biliary tree disease, alcoholic liver disease, or non-alcoholic steatohepatitis.

Embodiment 46. The method of any of embodiments 7-45, wherein the HSD17B13 inhibitor comprises a small molecule, a polypeptide, or an oligonucleotide.

Embodiment 47. The method of any of embodiments 7-46, wherein the HSD17B13 inhibitor comprises an estradiol mimetic, propyl pyrazole triole, an anti-HSD17B13 antibody or antibody fragment, an oligonucleotide directed against an HSD17B13 encoding oligonucleotide, an antisense oligonucleotide, an siRNA, ARO-HSD or ALN-HSD.

Embodiment 48. The method of any of embodiments 7-46, wherein the HSD17B13 inhibitor comprises:

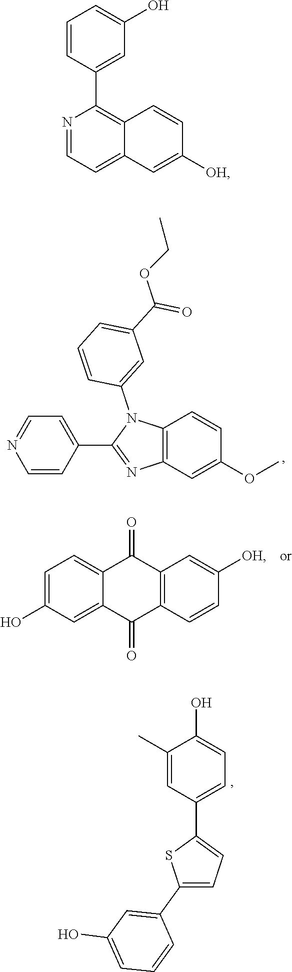

or a pharmaceutically acceptable salt or derivative thereof.

Embodiment 49. The method of any of embodiments 1-48, wherein measuring the amount of the reaction product or second amount of the HSD17B13 substrate comprises performing liquid chromatography, high-performance liquid chromatography or mass spectrometry.

Embodiment 50. The method of any of embodiments 1-49, wherein the sample comprises a blood sample, a plasma sample or a serum sample.

Embodiment 51. The method of any of embodiments 1-50, wherein the subject is a mammal.

Embodiment 52. The method of any of embodiments 1-51, wherein the subject is a human.

Embodiment 53. A kit for measuring an HSD17B13 enzyme activity, comprising:
a HSD17B13 substrate comprising LTB3, resolvin D1, resolvin E1, protectin DX, maresin 1, 12-HETE, 13-HODE, 15-HEDE, methyl malonyl coenzyme A or lauryl coenzyme A, or a salt thereof; and
a reagent for measuring a reaction product of the HSD17B13 substrate, or a reagent for measuring the HSD17B13 substrate.

Embodiment 54. The kit of embodiment 53, wherein the HSD17B13 substrate is labeled.

Embodiment 55. The kit of embodiment 53 or 54, wherein the HSD17B13 substrate is radiolabeled.

Embodiment 56. The kit of embodiment 55, wherein the radiolabeled HSD17B13 substrate comprises 13C or 2H.

Embodiment 57. The kit of any of embodiments 53-56, wherein the reaction product comprises an oxidized form of the HSD17B13 substrate.

Embodiment 58. The kit of any of embodiments 53-57, wherein the HSD17B13 substrate comprises a primary alcohol, and the reaction product comprises an aldehyde.

Embodiment 59. The kit of any of embodiments 53-57, wherein the HSD17B13 substrate comprises a secondary alcohol, and the reaction product comprises a ketone.

Embodiment 60. The kit of any of embodiments 53-59, further comprising a solution containing a known concentration of HSD17B13.

Embodiment 61. The kit of any of embodiments 53-60, further comprising instructions for using the reagent in an HSD17B13 activity assay.

Embodiment 62. An in-vivo modified protein comprising an HSD17B13 enzyme bound to a ligand, wherein the ligand comprises a labeled HSD17B13 substrate comprising LTB3, resolvin D1, resolvin E1, protectin DX, maresin 1, 12-HETE, 13-HODE, 15-HEDE, methyl malonyl coenzyme A or lauryl coenzyme A, or a salt thereof, or wherein the ligand comprises a labeled reaction product of the HSD17B13 substrate.

Embodiment 63. The in-vivo modified protein of embodiment 62, wherein the contact comprises a non-covalent bond between the HSD17B13 enzyme and the ligand.

Embodiment 64. The in-vivo modified protein of embodiment 63, wherein the non-covalent bond comprises a hydrogen bond, an ionic bond, a van der Waals interaction, or a hydrophobic bond.

Embodiment 65. The in-vivo modified protein of any of embodiments 62-64, wherein the HSD17B13 substrate is radiolabeled.

Embodiment 66. The in-vivo modified protein of embodiment 65, wherein the radiolabeled HSD17B13 substrate comprises 13C or 2H.

Embodiment 67. The in-vivo modified protein of any of embodiments 62-66, wherein the reaction product comprises an oxidized form of the HSD17B13 substrate.

Embodiment 68. The in-vivo modified protein of any of embodiments 62-67, wherein the HSD17B13 substrate comprises a primary alcohol, and the reaction product comprises an aldehyde.

Embodiment 69. The in-vivo modified protein of any of embodiments 62-67, wherein the HSD17B13 substrate comprises a secondary alcohol, and the reaction product comprises a ketone.

Embodiment 70. A ligand-HSD17B13 complex formed by binding a HSD17B13 enzyme to a ligand, the ligand comprising a labeled HSD17B13 substrate comprising LTB3, resolvin D1, resolvin E1, protectin DX, maresin 1, 12-HETE, 13-HODE, 15-HEDE, methyl malonyl coenzyme A or lauryl coenzyme A, or a salt thereof, or the ligand comprising a labeled reaction product of the HSD17B13 substrate.

Embodiment 71. The ligand-HSD17B13 complex of embodiment 70, wherein the contact comprises a non-covalent bond between the HSD17B13 enzyme and the ligand.

Embodiment 72. The ligand-HSD17B13 complex of embodiment 71, wherein the non-covalent bond comprises a hydrogen bond, an ionic bond, a van der Waals interaction, or a hydrophobic bond.

Embodiment 73. The ligand-HSD17B13 complex of any of embodiments 70-72, wherein the HSD17B13 substrate is radiolabeled.

Embodiment 74. The ligand-HSD17B13 complex of embodiment 73, wherein the radiolabeled HSD17B13 substrate comprises 13C or 2H.

Embodiment 75. The ligand-HSD17B13 complex of any of embodiments 70-74, wherein the reaction product comprises an oxidized form of the HSD17B13 substrate.

Embodiment 76. The ligand-HSD17B13 complex of any of embodiments 70-75, wherein the HSD17B13 substrate comprises a primary alcohol, and the reaction product comprises an aldehyde.

Embodiment 77. The ligand-HSD17B13 complex of any of embodiments 70-75, wherein the HSD17B13 substrate comprises a secondary alcohol, and the reaction product comprises a ketone.

Embodiment 78. A compound comprising a labeled HSD17B13 substrate comprising LTB3, resolvin D1, resolvin E1, protectin DX, maresin 1, 12-HETE, 13-HODE, 15-HEDE, methyl malonyl coenzyme A or lauryl coenzyme A, or a salt thereof.

Embodiment 79. The compound of embodiment 78, wherein the HSD17B13 substrate is radiolabeled.

Embodiment 80. The compound of embodiment 79, wherein the radiolabeled HSD17B13 substrate comprises 13C or 2H.

Embodiment 81. A compound comprising a reaction product of an HSD17B13 substrate comprising LTB3, resolvin D1, resolvin E1, protectin DX, maresin 1, 12-HETE, 13-HODE, 15-HEDE, methyl malonyl coenzyme A or lauryl coenzyme A, or a salt thereof.

Embodiment 82. The compound of embodiment 81, wherein the reaction product is labeled.

Embodiment 83. The compound of embodiment 81 or 82, wherein the reaction product is radiolabeled.

Embodiment 84. The compound of embodiment 83, wherein the radiolabeled reaction product comprises 13C or 2H.

Embodiment 85. The compound of any of embodiments 81-84, wherein the reaction product comprises an oxidized form of LTB3, resolvin D1, resolvin E1, protectin DX, maresin 1, 12-HETE, 13-HODE, 15-HEDE, methyl malonyl coenzyme A or lauryl coenzyme A, or the salt thereof.

Embodiment 86. The compound of any of embodiments 81-85, wherein the oxidized form of LTB3, resolvin D1, resolvin E1, protectin DX, maresin 1, 12-HETE, 13-HODE, 15-HEDE, methyl malonyl coenzyme A or lauryl coenzyme A, or the salt thereof comprises an aldehyde or ketone in place of a primary or secondary alcohol of LTB3, resolvin D1, resolvin E1, protectin DX, maresin 1, 12-HETE, 13-HODE, 15-HEDE, methyl malonyl coenzyme A or lauryl coenzyme A, or the salt thereof.

### EXAMPLES

The following examples are included for illustrative purposes only and are not intended to limit the scope of the invention.

### Example 1

Using a uniformly 13C-labeled 12-HETE orally to a subject and drawing plasma and using LC/MS-MS to measure the 13C-labeled 12-oxo-ETE/13C-labeled 12-HETE will provide a relative HSD17B13 activity. If this ratio is measured over time, this can be translated into an area under the curve to further refine the assessment (FIG. 2). FIG. 2 plots ratios of labeled product/labeled substrate over time following oral dosing of a labeled substrate. The ratio for the subject with active HSD17B13 is higher than the ratio observed for a subject with inactive or low HSD17B13 activity. It is anticipated that the ratio for subjects with inactive HSD17B13 may be undetectable.

### Example 2

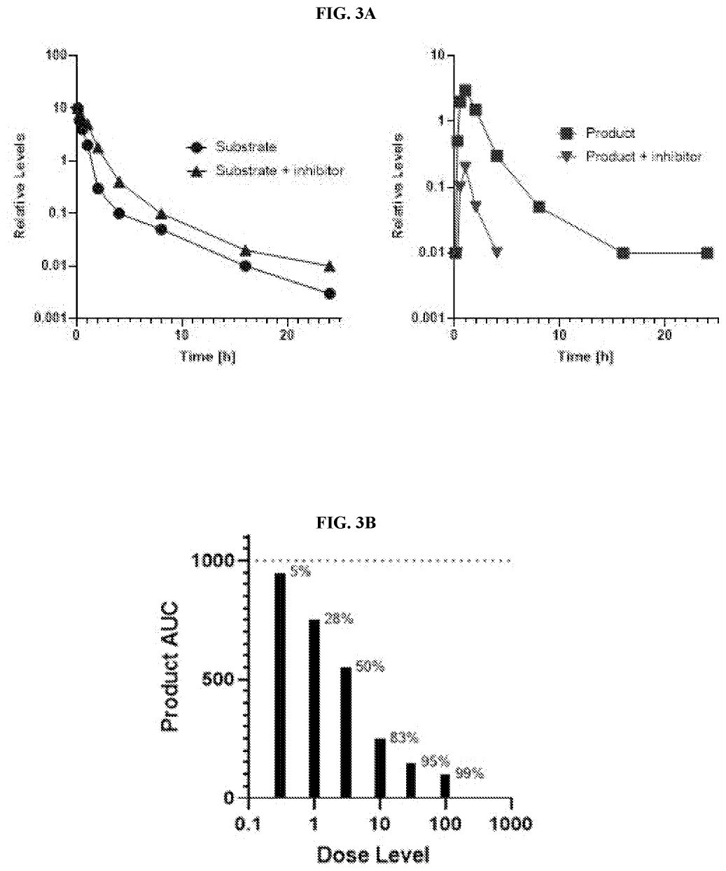

Using a uniformly 13C-labeled 12-HETE administered intravenously and using LC/MS-MS to measure plasma levels of both 13C-labeled 12-oxo-ETE product and the13C-labeled 12-HETE with or without dosing an HSD17B13 inhibitor. The extent of inhibition is determined by calculating the area under the curve for the labeled product over the time course. Doses that result in a 95% decrease in product AUC would be considered to be inhibiting HSD17B13 95% over the course of 24 hours as presented in FIG. 3A and FIG. 3B. FIG. 3A includes plots of labeled substrates and products following intravenous dosing of a subject with a labeled HSD17B13 substrate to measure an ability of an HSD17B13 inhibitor to inhibit oxidation of marker substrate. FIG. 3B shows an area under the curve for the product formed to assess percentage inhibition.

### Example 3—HSD17B13 Activity Assay Using NADH Fluorescence

A fluorescence based HSD17B13 activity assay was used to monitor the fluorescence of NADH, which is generated from NAD+ during the dehydrogenation of the substrate LTB3 or other substrates. Reactions were performed in a 384-well plates (Greiner; #655076) in a 20 μl reaction volume containing the following reagents (final concentrations): 3 mM NAD⁺ (Sigma; #N0623); 125 nM HSD17B13 enzyme (in-house E. coli expressed His-tagged, purified, soluble protein); 1 M potassium phosphate buffer, pH 7.4; 1% DMSO; and a range of substrate concentrations (2.5 nM-30 mM). Reactions proceeded for 1 hour at 26.5° C. Generation of NADH was measured as the fluorescence signal (Excitation at 340 nm and Emission at 445 nm) at 3 hours minus the baseline fluorescence at t=0. Fluorescence signals in the absence of added substrate (negative control values), were subtracted from all values so that the results reflected substrate-dependent production of NADH. NADH standards were included to allow the conversion of relative fluorescence units into rates of enzyme activity. Ten concentrations of substrate were tested (in the range of 30 mM-2.5 nM), and two independent assessments were performed at each concentration. Activity was compared to the positive control, LTB3, at a concentration of 25 mM. Results are reported as the ratio: [Activity of compound at 30 mM]/[Activity of LTB3 at 25 mM]. Data from this assay are included in Table 1b.

### Example 4: Substrates Identification for Pharmacodynamic Biomarkers

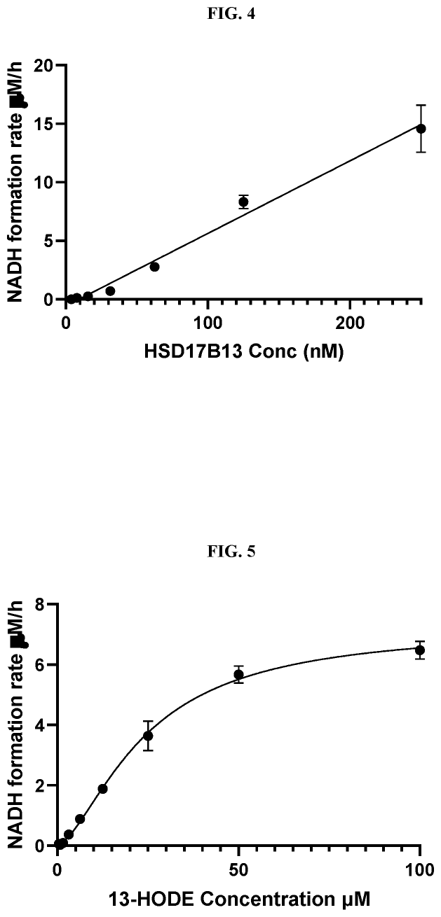

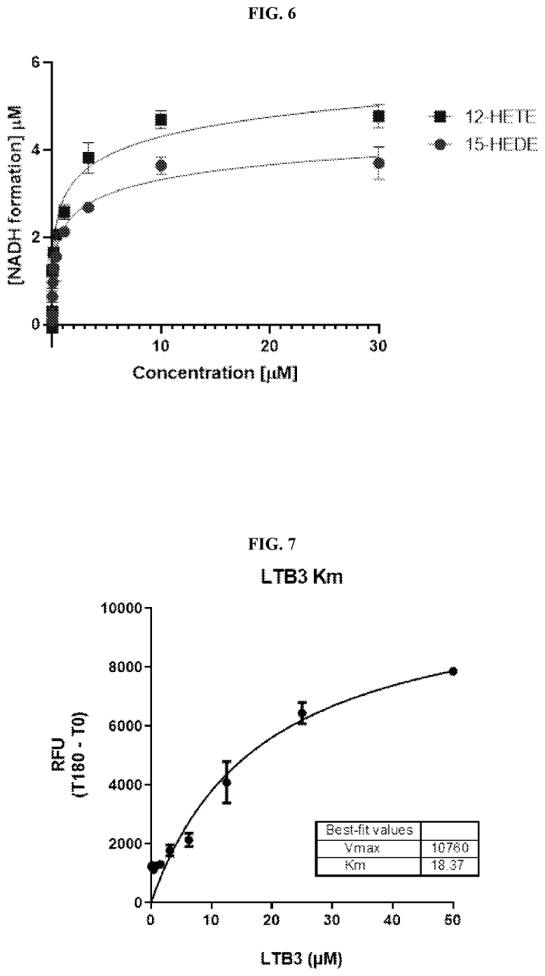

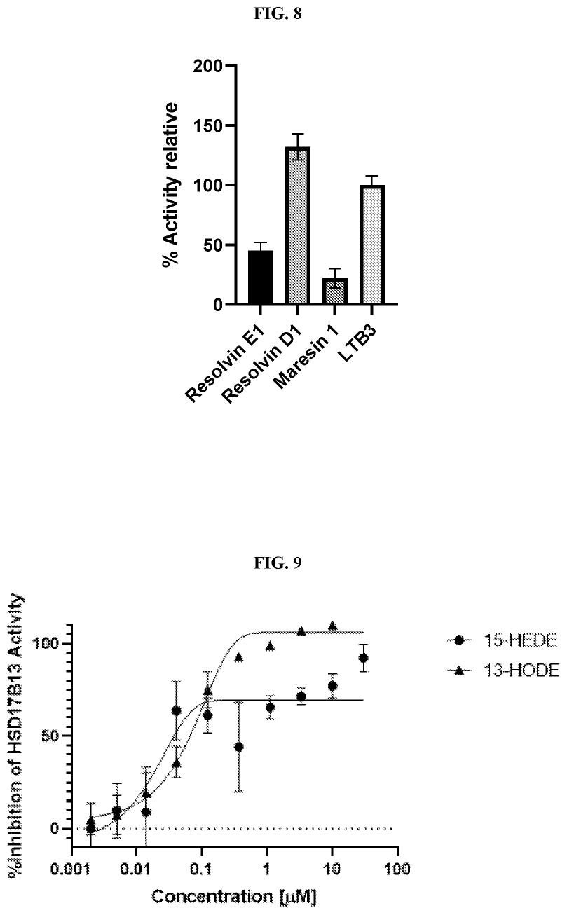

Enzyme reaction was performed in K₂PO₄ pH 7.4 buffer with final DMSO concentrations not exceeding 2%. The enzyme concentration was evaluated between 4 and 250 μM HSD17B13. Substrate turnover was evaluated at substrate concentrations ranging from 2 nM to 100 μM. The substrates evaluated include 12-HETE, 13-HODE,15-HEDE among others. The cofactor concentration was 3 mM NAD+ and was added to start the reaction. The reaction was allowed to proceed between 30-120 minutes. The production of NADH was quantified either by fluorescence or by bioluminescence. Data is illustrated in FIG. 4, FIG. 5, FIG. 6, FIG. 7, and FIG. 8.

### Example 5: Experimental for Measuring HSD17B13 Inhibition Using Marker Substrates Such as 13-HODE and 15-HEDE

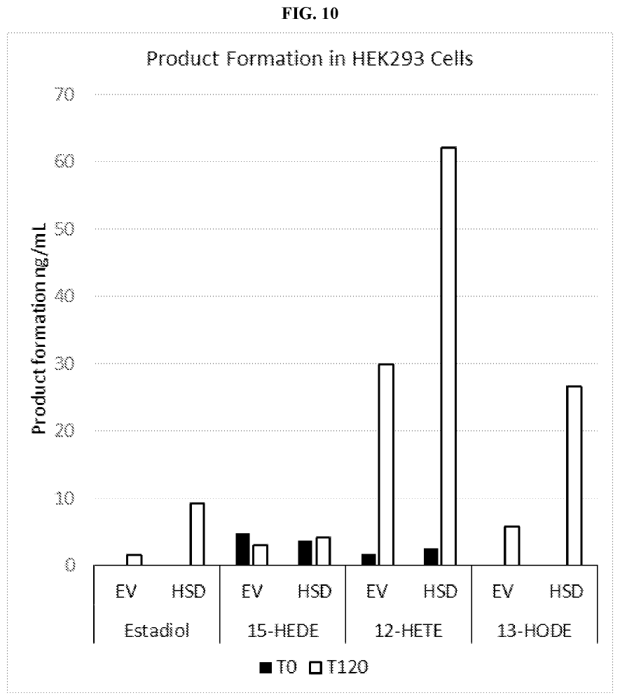

The ability of an HSD17B13 inhibitor to inhibit the substrate turnover was determined by measuring HSD17B13 enzyme activity on marker substrates. The HSD17B13 enzyme reaction was performed in K₂PO₄ pH 7.4 buffer with final DMSO concentrations not exceeding 2%. The 13-HODE turnover was evaluated at 45 μM 13-HODE and 125 nM HSD17B13. The 15-HEDE turnover was evaluated at 4.3 μM 15-HEDE and enzyme at 62.5 nM. The cofactor concentration was 3 mM NAD+ and was added to start the reaction. The reaction was allowed to proceed between 30-120 minutes. The production of NADH was quantified either by fluorescence or by bioluminescence. The percent inhibition was calculated based on the reaction in absence of inhibitor as 0% inhibited, and 100% inhibition was assumed to be equivalent to the reaction in the absence of substrate, as NADH formation was measured. Illustrated in FIG. 9.

### Example 6: Experimental for Confirming Selectivity of Marker Substrate Using a Cell-Based System with Transient Transfection for HSD17B13 Substrate Turnover

In order to confirm HSD17B13-specific substrate turnover, oxidative product formation from each substrate was evaluated by LC/MS following incubation of the substrate with HEK293 cells transiently transfected with either rHSD17B13 or an empty vector. 0.5×10\hat{ }6 cells were plated in 6 well plates followed by transfection with either empty vector or HSD17B13. The cells were then exposed to the test substrate at concentrations ranging from 10-300 μM in DMEM without phenol red culture media for time points ranging up to 180 minutes. Media samples are taken off of the cells and flowed through solid phase extraction cartridge. The solid phase cartridge is washed with 10% methanol in water and then the product is then eluted from the solid phase cartridge with 100% methanol. The oxidized product concentration is then determined by LC/MS quantitation. Illustrated in FIG. 10.

### Example 7: Experimental for Measuring Inhibition of HSD17B13-Dependent Oxidation of Marker Substrate in a Cell-Based System

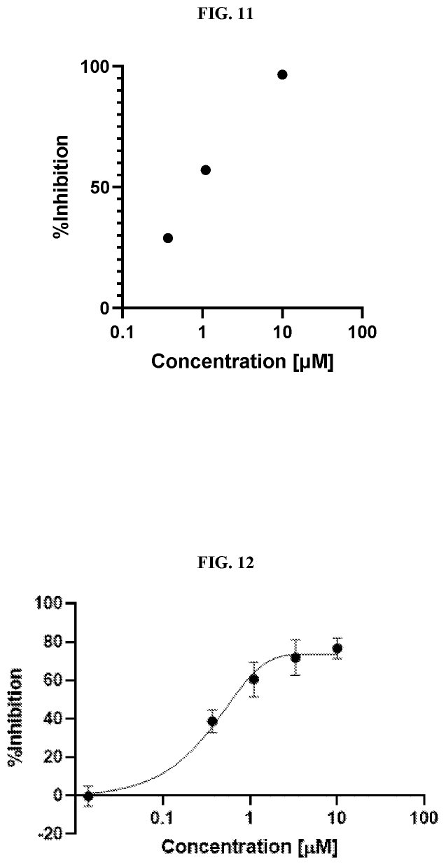

In order to confirm HSD17B13-specific inhibition of the marker substrate turnover, the oxidative product formation from the marker substrate was evaluated by LC/MS following incubation of the substrate with HEK293 cells transiently transfected with either rHSD17B13 or an empty vector. 0.5×10\hat{ }6 cells were plated in 6 well plates followed by transfection with either empty vector or HSD17B13. The cells were then exposed to the test substrate, 30 μM of 12-HETE or 13-HODE. Compound 1 was tested at concentrations ranging from 0.01 to 10 μM in DMEM without phenol red culture media for time points up to 180 minutes incubation with cells. Media samples were removed from the cells and flowed through solid phase extraction cartridge. The solid phase cartridge is washed with 10% methanol in water and then the product is then eluted from the solid phase cartridge with 100% methanol. The oxidized product concentration is then determined by LC/MS quantitation. The percent inhibition was calculated based on the % decrease in oxidized product formation in the media from HSD17B13-transfected cells-oxidized product formation in empty vector-transfected cells. This is illustrated in FIG. 11 and FIG. 12.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method of treatment, comprising:
administering to a subject a first amount of a 17β-Hydroxysteroid dehydrogenase type 13 (HSD17B13) substrate or substrate precursor;
determining an amount of a reaction product of the HSD17B13 substrate in a sample obtained from the subject, or determining a second amount of the HSD17B13 substrate or substrate precursor in a sample obtained from the subject, after administering the first amount of the HSD17B13 substrate or substrate precursor to the subject;
determining an HSD17B13 enzyme activity based on the amount of the reaction product, the second amount of the HSD17B13 substrate, or the second amount of the HSD17B13 substrate precursor in the sample; and determining an amount of the HSD17B13 inhibitor to administer to the subject based on the HSD17B13 enzyme activity; and
administering or co-administering a first amount of an HSD17B13 inhibitor to the subject with the administration of the first amount of the HSD17B13 substrate or substrate precursor;
wherein the reaction product comprises an oxidized form of the HSD17B13 substrate;
the HSD17B13 substrate comprises LTB3, resolvin D1, resolvin E1, protectin DX, maresin 1, 12-HETE, 15-HETE, 13-HODE, 15-HEDE, methyl malonyl coenzyme A, or lauryl coenzyme A, or a salt thereof; and
the HSD17B13 substrate precursor comprises arachidonic acid or linoleic acid, or a salt thereof.

2-5. (canceled)

6. The method of claim 1, wherein the sample is obtained from the subject about 1 min, about 5 min, about 10 min, about 15 min, about 20 min, about 25 min, about 30 min, about 35 min, about 40 min, about 45 min, about 50 min, about 55 min, about 1 hr, about 2 hr, about 4 hr, about 6 hr, about 8 hr, about 10 hr, about 12 hr, about 14 hr, about 16 hr, about 18 hr, about 20 hr, about 22 hr, about 24 hr, or longer, after administering the first amount of the HSD17B13 substrate or substrate precursor to the subject.

7-8. (canceled)

9. The method of claim 1, wherein the administration of the first amount of the HSD17B13 substrate or substrate precursor is intravenous or oral.

10. The method of claim 1, wherein the first amount of the HSD17B13 substrate or substrate precursor is 10-1000 μg of the HSD17B13 substrate or substrate precursor, wherein the first amount of the HSD17B13 substrate or substrate precursor is optionally about 100 μg.

11. (canceled)

12. The method of claim 1, wherein the HSD17B13 substrate or substrate precursor and the HSD17B13 reaction product are labeled, which optionally comprise ¹³C or ²H.

13-14. (canceled)

15. The method of claim 12, wherein determining the amount of the reaction product comprises measuring the labeled reaction product in the sample, or determining the second amount of the HSD17B13 substrate or substrate precursor comprises measuring the labeled HSD17B13 substrate or substrate precursor in the sample.

16-17. (canceled)

18. The method of claim 1, further comprising determining a second amount of the reaction product of the HSD17B13 substrate or substrate precursor in a second sample, or determining a third amount of the HSD17B13 substrate or substrate precursor in the second sample, wherein the second sample is obtained from the subject at a second time after administering the first amount of the HSD17B13 substrate to the subject wherein the second time comprises about 2 min, about 5 min, about 10 min, about 15 min, about 20 min, about 25 min, about 30 min, about 35 min, about 40 min, about 45 min, about 50 min, about 55 min, about 1 hr, about 2 hr, about 4 hr, about 6 hr, about 8 hr, about 10 hr, about 12 hr, about 14 hr, about 16 hr, about 18 hr, about 20 hr, about 22 hr, about 24 hr, or longer.

19. (canceled)

20. The method of claim 18, further comprising comparing the second amount of the reaction product to the first amount of the reaction product, comparing the third amount of the HSD17B13 substrate to the second amount of the HSD17B13 substrate, or comparing the third amount of the HSD17B13 substrate precursor to the second amount of the HSD17B13 substrate precursor, wherein optionally generating an area under the curve of the first and second amounts of the reaction product, generating an area under the curve of the second and third amounts of the HSD17B13 substrate, or generating an area under the curve of the second and third amounts of the HSD17B13 substrate precursor.

21. (canceled)

22. The method of claim 20, further comprising comparing a first area under the curve of the first and second amounts of the reaction product, or the second and third amounts of the HSD17B13 substrate or substrate precursor, to a second area under the curve of amounts of the reaction product, HSD17B13 substrate, or substrate precursor, wherein the first area under the curve is generated when the HSD17B13 substrate or substrate precursor is coadministered to the subject with an HSD17B13 inhibitor, and wherein the second area under the curve is generated when the HSD17B13 substrate or substrate precursor is not coadministered to the subject with the HSD17B13 inhibitor.

23. The method of claim 20, further comprising comparing a first area under the curve of the first and second amounts of the reaction product, or the second and third amounts of the HSD17B13 substrate or substrate precursor, to a second area under the curve of amounts of the reaction product or HSD17B13 substrate or substrate precursor, wherein the first area under the curve is generated when the HSD17B13 substrate or substrate precursor is coadministered to the subject with a first amount of an HSD17B13 inhibitor, and wherein the second area under the curve is generated when the HSD17B13 substrate or substrate precursor is coadministered to the subject with a second amount of the HSD17B13 inhibitor.

24. The method of claim 23, further comprising comparing the HSD17B13 enzyme activity to a baseline HSD17B13 enzyme activity obtained without co-administering the HSD17B13 inhibitor to the subject with the HSD17B13 substrate or substrate precursor, and determining based on the comparison whether to increase, decrease, or not change the second amount of the HSD17B13 inhibitor relative to the first amount of the HSD17B13 inhibitor.

25-29. (canceled)

26. The method of claim 1, further comprising administering, with the first amount of the HSD17B13 substrate or substrate precursor, a first amount of an HSD17B13 inhibitor, wherein the first amount of the HSD17B13 inhibitor inhibits reaction product formation by about 25%, about 50%, about 75%, about 85%, about 90%, or about 95%.

31. (canceled)

32. The method of claim 30, further comprising, based on the determined amount of HSD17B13 inhibitor that inhibits reaction product formation by about 25%, about 50%, about 75%, about 85%, about 90%, or about 95%, identifying a dose of the HSD17B13 inhibitor to administer to the subject for treatment.

33. The method of claim 1, wherein the dose of the HSD17B13 inhibitor is sufficient to treat a liver disease in the patient, wherein the liver disease comprises liver inflammation, liver fibrosis, cholestasis, a gall bladder disease, a biliary tree disease, alcoholic liver disease, or non-alcoholic steatohepatitis.

34. The method of claim 1, further comprising normalizing the HSD17B13 enzyme activity to obtain a specific HSD17B13 enzyme activity of the subject, wherein further comprising comparing the HSD17B13 enzyme activity of the subject to a control HSD17B13 enzyme activity measurement, a baseline HSD17B13 enzyme activity measurement, or a threshold HSD17B13 enzyme activity, further comprising identifying the subject as likely to benefit from the treatment with the HSD17B13 inhibitor when the HSD17B13 enzyme activity of the subject is greater than the control HSD17B13 enzyme activity measurement, baseline HSD17B13 enzyme activity measurement, or threshold HSD17B13 enzyme activity.

35-43. (canceled)

44. The method of claim 1, further comprising identifying the subject as likely to develop the liver disease when the HSD17B13 enzyme activity of the subject is greater than the control HSD17B13 enzyme activity measurement, baseline HSD17B13 enzyme activity measurement, or threshold HSD17B13 enzyme activity.

45. (canceled)

46. The method of claim 1, wherein the HSD17B13 inhibitor comprises a small molecule, a polypeptide, or an oligonucleotide.

47. The method of claim 1, wherein the HSD17B13 inhibitor comprises an estradiol mimetic, propyl pyrazole triole, an anti-HSD17B13 antibody or antibody fragment, an oligonucleotide directed against an HSD17B13 encoding oligonucleotide, an antisense oligonucleotide, an siRNA, ARO-HSD or ALN-HSD.

48. The method of claim 1, wherein the HSD17B13 inhibitor is: 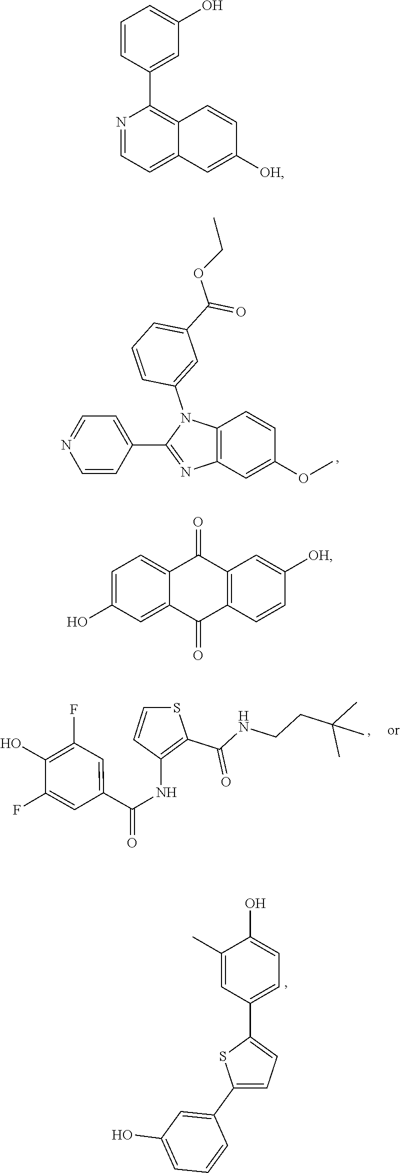
or a pharmaceutically acceptable salt or derivative thereof.

49-55. (canceled)

56. The method of claim 1, wherein the sample comprises a blood sample, a plasma sample or a serum sample, and the subject is a mammal.

57-92. (canceled)
